(19) United States
(12) Patent Application Publication
(10) Pub. No.: US 2008/0312209 A1
(43) Pub. Date: Dec. 18, 2008

(54) Piperazine Heteroaryl Derivatives as Gpr38 Agonists

(75) Inventors: Gregor James MacDonald, Essex (GB); Steven James Stanway, Essex (GB); Mervyn Thompson, Essex (GB); Susan Marie Westaway, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 11/995,416

(22) PCT Filed: Jul. 12, 2005
(86) PCT No.: PCT/GB2005/002731

§ 371 Date: Jun. 9, 2008

(51) Int. Cl. A61K 31/496; C07D 401/14; C07D 417/08; A61P 1/00

(52) U.S. Cl. 514/210.18; 544/364; 514/253.13; 544/369; 514/254.02

(57) ABSTRACT

The invention provides compounds of formula (I) or pharmaceutically acceptable salts thereof,

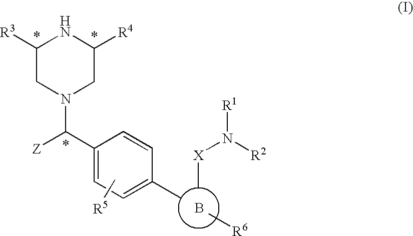

wherein R¹, R², R³, R⁴, R⁵, R⁶, Z, X and B are as defined in the specification. The compounds are partial or full agonists at the GPR38 receptor. Pharmaceutical compositions comprising the compounds, methods of preparing the compounds, uses of the compounds and methods involving the compounds are also provided.

The present invention relates to novel bi-aryl derivatives and related compounds, having pharmaceutical activity, processes for their preparation, pharmaceutical compositions containing them and to their use in the treatment of various disorders.

GPR38 is a 7-transmembrane, G-protein coupled receptor, with high affinity for the peptide motilin [Feighner et al., Science 1999, 284, 2184], suggesting that endogenous motilin exerts all or most of its activity via this receptor.

Motilin is a 22 amino acid peptide found in large amounts within endocrine-like cells of the gastrointestinal tract, and especially in the duodenum-jejunum areas. During fasting, the peptide is known to be associated with the onset of Phase III migrating complex activity within the stomach [Boivin et al., Dig. Dis. Sci. 1992, 37, 1562], suggesting a role in the mechanisms of this prokinetic activity. Motilin is also released from the gut during feeding, sham feeding, gastric distension or by oral or intravenous nutrient application [Christofides et al., Gut 1979, 20, 102; Bormans et al., Scand. J. Gastroenterol. 1987, 22, 781], suggesting additional roles for this peptide in the modulation of motility patterns during feeding.

In animals or in man, motilin has long been known to increase gastrointestinal motility, and promote gastric emptying and intestinal propulsion in an anal direction, during both fasting and fed conditions. This activity is thought to be primarily due to a facilitation of at least the cholinergic excitatory function of the gut [Van Assche et al., Eur. J. Pharmacol. 1997, 337, 267], perhaps also involving the activation of the vagus nerve [Mathis & Malbert, Am. J. Physiol. 1998, 274, G80]. In addition, higher concentrations of motilin directly evoke a small contraction of the muscle [Van Assche et al., Eur. J. Pharmacol. 1997, 337, 267].

The antibiotic erythromycin was shown to mimic the gastrointestinal activity of motilin, in addition to its previously-described antibiotic properties [see Peeters, in Problems of the Gastrointestinal Tract in Anaesthesia Ed., Herbert M K et al. Springer-Verlag, Berlin, Heidelberg 1999, pp 39-51]. More recently, erythromycin has been shown to activate the GPR38 receptor, confirming its ability to mimic the function of motilin [Carreras et al., Analyt. Biochem. 2002, 300, 146]. In addition, the availability of this non-peptide motilin receptor agonist has allowed at least some clinical studies to be undertaken in order to examine the clinical potential of motilin receptor agonists. These studies have consistently demonstrated an ability to increase gastric emptying in various conditions associated with gastroparesis, such as functional dyspepsia and diabetic gastroparesis. Further, erythromycin has been shown to increase lower esophageal sphincter pressure in man, which together with the increase in gastric emptying, suggests a role in the treatment of gastroesophageal reflux disease (GERD). Finally, erythromycin has been used to promote intestinal propulsive activity, finding clinical utility in the treatment of pseudo-obstruction and in conditions with impaired colonic motility [Peeters, in Problems of the Gastrointestinal Tract in Anaesthesia Ed., Herbert M K et al. Springer-Verlag, Berlin, Heidelberg 1999, pp 39-51].

Consequently it is expected that agonists at the GPR38 receptor will mimic the activity of motilin and find clinical utility in the treatment of gastrointestinal disorders associated with hypomotility, especially the functional bowel disorders such as GERD, functional dyspepsia (FD) and irritable bowel syndrome (IBS). The compounds will also be useful for the treatment of other GI conditions where the cause is known and in which GI motility is reduced. Such conditions include constipation, caused by various diseases such as those associated with neuropathy, and/or by the administration of other drugs, intestinal pseudo-obstruction, paralytic ileus following surgery or some other manipulation, gastric stasis or hypomotility caused by various diseases such as diabetes and/or by the administration of other drugs. Interestingly, the ability of motilin or erythromycin to activate the vagus nerve, the association of this nerve with changes in feeding behaviour [eg. Furness et al., Auton. Neurosci. 2001, 92, 28] and the chromosomal location of GPR38 [based on Ensembl: 13q21.1 (58.46-59.46 Mb)] within the markers (D13S257-13q14.11 to D13S258 at 13q21.33) of a locus associated with obesity [Feitosa et al, Am. J. Hum. Genet. 2002, 70, 72] also suggests that agonists active at the GPR38 receptor will, in addition to promoting gastrointestinal motility, facilitate eating behaviours in at least those patients in which some degree of appetite suppression or cachexia is present. Such activity indicates that agonists at this receptor will find clinical utility in the treatment of symptoms associated with—for example—the treatment of cancer or by the presence of the cancer itself.

In addition to the ability of motilin receptor agonists to promote gastrointestinal motility, the association of motilin gene polymorphism with Crohn's disease [Annese et al., Dig. Dis. Sci. 1998, 43, 715-710] and the changes in motilin receptor density during colitis [Depoortere et al., Neurogastroenterol. Motil. 2001, 13, 55] suggests a utility for agonists at the motilin receptor for the treatment of inflammatory bowel conditions in general.

Finally, GPR38 is also found in regions outside the gastrointestinal tract. These areas include the pituitary, adipose tissue, urinary bladder and certain areas of the brain. The former suggests clinical utility in the promotion of pituitary function, such as the release of growth hormone secretagogues, the presence within adipose tissue again suggests a role in the control of body weight, and the presence within the urinary bladder suggests a role for agonists at this receptor in the treatment of incontinence. The presence of GPR38 within the brain supports the gastrointestinal and feeding utilities already mentioned, but in addition, suggests an involvement of the receptor in a greater spectrum of vagal-hypothalamic functions.

Patents WO9410185, EP838469, WO9823629, DE19805822, and U.S. Pat. No. 6,165,985 claim erythromycin derivatives targetting GPR³⁸ for use in disorders relating to gastrointestinal motility. Patents WO9921846, WO0185694, WO0168620, WO0168621, and WO0168622 disclose a series of small molecule antagonists of the GPR³⁸ receptor. Patents JP07138284 and EP807639 disclose peptide agonists. JP09249620 and WO02092592 disclose a series of small molecule agonists.

A structurally novel class of compounds has now been found which are partial or full agonists at the GPR³⁸ receptor.

In a first aspect, the present invention therefore provides a compound of formula (I) or a pharmaceutically acceptable salt thereof, wherein

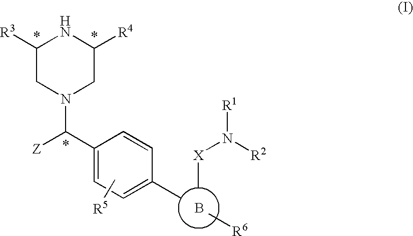

X is CH₂, CO or SO₂

R³ and R⁴ are independently H or C₍₁₋₄₎alkyl;

R¹ is C₍₁₋₄₎alkyl;

R² is YR⁷;

or R¹ and R² together with the nitrogen to which they are attached form a 4, 5, 6 or 7 membered heterocyclic ring, optionally substituted with one or more substituents independently selected from C₍₁₋₄₎alkyl, hydroxy, ═O or WR⁷;

Y is CO(CH₂)ₙ, SO₂(CH₂)ₙ, (CH₂)ₙ, (CH₂)ₙA, CO(CH₂)ₙA, SO₂(CH₂)ₙA where n is 1, 2, 3 or 4 and A is O, S, CO, SO₂, NH, NHCO, CONH; or N—C₍₁₋₄₎alkyl

W is a bond, CH₂, O, S, CO, SO₂, NH, NHCO, CONH or N—C₍₁₋₄₎alkyl

R⁷ is optionally substituted phenyl, an optionally substituted 5 or 6 membered heterocyclic ring or an optionally substituted 5 or 6 membered heteroaryl ring;

R⁵ is hydrogen, halogen, or C₍₁₋₄₎alkoxy;

R⁶ is hydrogen, halogen, C₍₁₋₄₎alkyl or C₍₁₋₄₎alkoxy;

Z is H or C₍₁₋₄₎alkyl;

B is a 5 or 6 membered heteroaryl;

and when R⁷ is substituted, it may have 1, 2 or 3 substituents, each independently selected from halogen, C₍₁₋₄₎alkyl, C₍₁₋₄₎alkoxy C₃₋₇cycloalkyl, hydroxy, trifluoromethoxy, trifluoromethyl, nitro, cyano, phenyl, NH₂, NHR⁸, NR⁸R⁹, C(O)CF₃, C(O)C₁₋₄alkyl, C(O)C₃₋₇cycloalkyl, CONH₂, CONHR⁸, CONR⁸R⁹, SOR⁹, SO₂R⁹, OSO₂R⁹, OSO₂CF₃, SO₂NH₂, SO₂NHR⁸, SO₂NR⁸R⁹, where R⁸ and R⁹═C₍₁₋₄₎alkyl, phenyl optionally substituted with halogen or 5 or 6 membered heteroaryl optionally substituted with halogen.

The term “alkyl” as a group or part of a group e.g. alkoxy or hydroxyalkyl refers to a straight or branched alkyl group in all isomeric forms. Examples of such alkyl groups include methyl, ethyl, propyl, isopropyl, butyl, isobutyl, sec-butyl, tert-butyl, pentyl, neopentyl, sec-pentyl, n-pentyl, isopentyl, tert-pentyl, hexyl and heptyl. Particular examples of such alkyl groups for R¹ include methyl, ethyl, propyl, isopropyl, butyl, sec-butyl, tert-butyl, pentyl, neopentyl, sec-pentyl, n-pentyl, isopentyl, tert-pentyl, hexyl and heptyl. Examples of such alkoxy groups include methoxy, ethoxy, propoxy, isopropoxy, butoxy, isobutoxy, sec-butoxy, tert-butoxy, pentoxy, neopentoxy, sec-pentoxy, n-pentoxy, isopentoxy, tert-pentoxy and hexoxy.

As used herein, the term “halogen” refers to fluorine (F), chlorine (Cl), bromine (Br), or iodine (I) and the term “halo” refers to the halogen fluoro (—F), chloro (—Cl), bromo(—Br), and iodo(—I).

The term “heteroaryl” represents a 5 or 6 membered unsaturated ring which comprises one or more heteroatoms. When the term heteroaryl represents a 5 membered group it contains a heteroatom selected from O, N or S and may optionally contain a further 1 to 3 nitrogen atoms. When heteroaryl represents a 6-membered group it contains from 1 to 3 nitrogen atoms. Examples of such 5 or 6 membered heteroaryl rings include, pyrrolyl, triazolyl, isothiazolyl, thiadiazolyl, tetrazolyl, imidazolyl, pyrazolyl, thiazolyl, isoxazolyl, oxazolyl, oxadiazolyl, furazanyl, furanyl, thienyl, pyridyl, pyrimidinyl, pyrazinyl, pyridazinyl and triazinyl.

The term “4, 5, 6 or 7 membered heterocyclic ring” refers to a heterocyclic ring having 4, 5, 6 or 7 atoms in total. A heterocyclic ring may, for example, be at least partially saturated. A heterocyclic ring may be saturated. When the heterocyclic ring is formed by NR¹R², R¹ and R² together represent a 3, 4, 5 or 6 membered chain consisting of either 3, 4, 5 or 6 carbon members each linked by a single bond or 2, 3, 4 or 5 carbon members and an additional member selected from N, O or S each linked by a single bond and wherein the carbon members may be substituted by one or more substituents independently selected from C₍₁₋₄₎alkyl, such as methyl, hydroxy, ═O, or WR⁷. When the additional member is N, it may, for example, be present as an NH group or as an N—Cl-alkyl group. The term “5 or 6 membered heterocyclic ring” is to be interpreted in analogous fashion. When B is a heterocyclic ring, it may optionally contain one further heteroatom selected from nitrogen, oxygen or sulfur. In either case, when the additional member or further heteroatom is nitrogen then this may be present as NH or an N-substituted derivative thereof e.g. N-alkyl, N-acyl. When the additional member or further heteroatom is sulphur this may also be present as the SO, SO₂. Examples of 5 or 6 membered heterocyclic rings include pyrrolidinyl, piperidinyl, piperazinyl, morpholinyl or thiomorpholinyl. When R⁷ represents a heterocyclic ring it may contain one or more heteroatoms independently selected from nitrogen, oxygen or sulfur. When a heteroatom is nitrogen then this may be present as NH or an N-substituted derivative thereof e.g. N-alkyl, N-acyl. When a heteroatom is sulphur this may also be present as the SO, SO₂. Examples of such 5 or 6 membered heterocyclic rings include pyrrolidinyl, piperidinyl, piperazinyl, morpholinyl thiomorpholinyl, imidazolidinyl, pyrazolidinyl, oxazolidinyl, isothiazolidinyl, thiazolidinyl, dioxolanyl, thiazinanyl, dioxanyl, tetrahydrofuranyl, dithianyl and pyranyl.

In one embodiment, R¹ and R² together with the nitrogen to which they are attached form a 5 or 6 membered heterocyclic ring, optionally substituted with one or more substituents independently selected from C₍₁₋₄₎alkyl, hydroxy, ═O or WR⁷

Where R¹ and R² together with the nitrogen to which they are attached form an optionally substituted heterocyclic ring, when there is more than one substituent present, there may be two substituents at one position on the heterocyclic ring.

In one embodiment, the phenyl ring and the group XNR¹R² are on adjacent atoms in the B ring.

In one embodiment, R⁵ is hydrogen or halogen.

When R⁷ is substituted, it may have 1, 2 or 3 substituents, each independently selected from halogen, C₍₁₋₄₎alkyl, C₍₁₋₄₎alkoxy, cyano, CONH₂, CONHR⁸, CONR⁸R⁹, SO₂NH₂, SO₂NHR⁸, SO₂NHR⁸R⁹ where R⁸ and R⁹═C₍₁₋₄₎alkyl or optionally substituted phenyl or heteroaryl

When R¹ and R² together with the nitrogen to which they are attached form a 4, 5, 6 or 7 membered heterocyclic ring e.g. azetidinyl, pyrrolidinyl, piperidinyl or homopiperidinyl, it is, for example, substituted with one or more substituents, one of which is WR⁷.

In one embodiment of the invention when X is CH₂ then R² is the group YR⁷ wherein Y and R⁷ have the meanings defined above. Exemplary Y groups include CO(CH₂)ₙ e.g. CO(CH₂)₂ or CO(CH₂)ₙA e.g. CO(CH₂)ₙO. Exemplary R⁷ groups include phenyl or substituted phenyl e.g. halophenyl, for example fluorophenyl, difluorophenyl or chlorophenyl. In one embodiment, Y is CO(CH₂)ₙ or CO(CH₂)ₙA, and R⁷ is optionally substituted phenyl.

In a second embodiment of the invention when X is CO or SO₂ then R¹ and R² together with the nitrogen to which they are attached form a 4, 5, 6 or 7 membered heterocyclic ring, optionally substituted with one or more substituents, one of which is WR⁷. For example, R¹ and R² together with the nitrogen to which they are attached form a 4, 5 or 6 membered heterocyclic ring; for example a 5 or 6 membered heterocyclic ring.

In one embodiment, X is CO.

In a further embodiment, R¹ and R² together with the nitrogen to which they are attached form a 5 or 6 membered heterocyclic ring e.g. pyrrolidinyl or piperidinyl, substituted with one or more substituents, one of which is WR⁷. WR⁷ may be as defined above, but W is, for example, a bond, CH₂, NH, O or CO. W is, for example, NH or CH₂. For example, R⁷ is optionally substituted phenyl, e.g. phenyl or phenyl substituted by one or more groups selected from halogen (for example fluorine or chlorine), cyano, methoxy or CONH₂. Preferably, R⁷ is optionally substituted phenyl, e.g. phenyl or phenyl substituted by one or more groups selected from halogen, cyano or CONH₂. Within this embodiment NR¹R² conveniently represents piperidinyl.

In a further embodiment, R¹ is methyl and R² is YR⁷ where Y is (CH₂)ₙA or CO(CH₂)ₙA; n is, for example, 1 or 2 and A is, for example, O or CH₂

In a further embodiment, B is, for example, thiazolyl, pyrazolyl imidazolyl or pyridinyl, for example thiazolyl, pyrazolyl or pyridinyl.

In a further embodiment R³ and R⁴ each represent hydrogen or methyl, for example methyl. In one embodiment, at least one of R³ and R⁴ does not represent hydrogen.

In a further embodiment R⁵ represents hydrogen. In a further embodiment, R⁶ represents hydrogen.

In a further embodiment, Z represents hydrogen.

In certain of the compounds of formula (I), dependent upon the nature of the substituent there are chiral carbon atoms, such as the carbon atom marked with an “*”, and therefore compounds of formula (I) may exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses. Exemplary compounds of formula (I) wherein R³ and R⁴ are other than hydrogen e.g. methyl are those wherein the piperazine C* carbons have the 3R,5S-configuration.

Certain of the compounds herein can exist in various tautomeric forms and it is to be understood that the invention encompasses all such tautomeric forms.

Exemplary compounds of the invention are:
(3R,5S)-3,5-dimethyl-1-({4-[2-({4-[(4-fluorophenyl)methyl]piperidin-1-yl}carbonyl)pyridin-3-yl]phenyl}methyl)piperazine (E1)
(3R,5S)-3,5-dimethyl-1-({4-[2-({4-[(4-fluorophenyl)amino]piperidin-1-yl}carbonyl)pyridin-3-yl]phenyl}methyl)piperazine (E2)
N-{[3-(4-{[(3R,5S)-3,5-dimethyl-1-piperazinyl]methyl}phenyl)-2-pyridinyl]methyl}-3-(4-fluorophenyl)-N-methylpropanamide (E3)
(3R,5S)-3,5-dimethyl-1-({4-[4-({4-[(4-fluorophenyl)methyl]piperidin-1-yl}carbonyl)-2-methyl-1,3-thiazol-5-yl]phenyl}methyl)piperazine (E4)
(3R,5S)-3,5-dimethyl-1-({4-[4-({4-[(4-fluorophenyl)amino]piperidin-1-yl}carbonyl)-2-methyl-1,3-thiazol-5-yl]phenyl}methyl)piperazine (E5)
N-{[5-(4-{[(3R,5S)-3,5-dimethyl-1-piperazinyl]methyl}phenyl)-2-methyl-1,3-thiazol-4-yl]methyl}-3-(4-fluorophenyl)-N-methylpropanamide dihydrochloride (E6)
N-{[5-(4-{[(3R,5S)-3,5-dimethyl-1-piperazinyl]methyl}phenyl)-2-methyl-1,3-thiazol-4-yl]methyl}-2-[(4-fluorophenyl)oxy]-N-methylacetamide (E7)
N-(4-fluorophenyl)-1-{[3-(4-{[(3S)-3-methyl-1-piperazinyl]methyl}phenyl)-2-pyridinyl]carbonyl}-4-piperidinamine (E8)
1-{[3-(4-{[(3R,5S)-3,5-dimethyl-1-piperazinyl]methyl}-3-fluorophenyl)-2-pyridinyl]carbonyl}-N-(4-fluorophenyl)-4-piperidinamine (E9)
1-{[3-(4-{[(3R,5S)-3,5-dimethyl-1-piperazinyl]methyl}phenyl)-2-pyridinyl]carbonyl}-N-(3-fluorophenyl)-4-piperidinamine (E10)
1-{[3-(4-{[(3R,5S)-3,5-dimethyl-1-piperazinyl]methyl}-3-fluorophenyl)-2-pyridinyl]carbonyl}-N-(3-fluorophenyl)-4-piperidinamine (E11)
N-(3,4-difluorophenyl)-1-{[3-(4-{[(3R,5S)-3,5-dimethyl-1-piperazinyl]methyl}-3-fluorophenyl)-2-pyridinyl]carbonyl}-4-piperidinamine (E12)
(3R,5S)-1-({4-[2-({4-[(4-chlorophenyl)thio]-1-piperidinyl}carbonyl)-3-pyridinyl]-2-fluorophenyl}methyl)-3,5-dimethylpiperazine (E13)
N-(3-fluorophenyl)-1-{[3-(4-{[(3S)-3-methyl-1-piperazinyl]methyl}phenyl)-2-pyridinyl]carbonyl}-4-piperidinamine (E14)
N-(3,4-difluorophenyl)-1-{[3-(4-{[(3R,5S)-3,5-dimethyl-1-piperazinyl]methyl}phenyl)-2-pyridinyl]carbonyl}-4-piperidinamine (E15)
1-{[3-(4-{[(3R,5S)-3,5-dimethyl-1-piperazinyl]methyl}phenyl)-2-pyridinyl]carbonyl}-N-(4-fluorophenyl)-3-pyrrolidinamine (E16)
1-({3-[4-{[(3R,5S)-3,5-dimethyl-1-piperazinyl]methyl}-3-(methyloxy)phenyl]-2-pyridinyl}carbonyl)-N-(4-fluorophenyl)-4-piperidinamine (E17)
(3R,5S)-1-({4-[2-({(3S)-3-[(4-fluorophenyl)oxy]-1-pyrrolidinyl}carbonyl)-3-pyridinyl]phenyl}methyl)-3,5-dimethylpiperazine (E18)
(3R,5S)-1-({4-[2-({(3R)-3-[(4-fluorophenyl)oxy]-1-pyrrolidinyl}carbonyl)-3-pyridinyl]phenyl}methyl)-3,5-dimethylpiperazine (E19)
1-{[3-(4-{[(3R,5S)-3,5-dimethyl-1-piperazinyl]methyl}-3-fluorophenyl)-2-pyridinyl]sulfonyl}-N-(2-fluorophenyl)-4-piperidinamine (E20)
1-{[1-(4-{[(3R,5S)-3,5-dimethyl-1-piperazinyl]methyl}phenyl)-1H-imidazol-2-yl]carbonyl}-N-(3-fluorophenyl)-4-piperidinamine (E21)
1-{[1-(4-{[(3R,5S)-3,5-dimethyl-1-piperazinyl]methyl}phenyl)-1H-imidazol-2-yl]carbonyl}-N-(4-fluorophenyl)-4-piperidinamine (E22)
1-{[1-(4-{[(3R,5S)-3,5-dimethyl-1-piperazinyl]methyl}phenyl)-1H-imidazol-2-yl]carbonyl}-N-(2-fluorophenyl)-4-piperidinamine (E23)
1-{[1-(4-{[(3R,5S)-3,5-dimethyl-1-piperazinyl]methyl}-3-fluorophenyl)-1H-imidazol-2-yl]carbonyl}-N-(4-fluorophenyl)-4-piperidinamine (E24)
1-{[1-(4-{[(3R,5S)-3,5-dimethyl-1-piperazinyl]methyl}-3-fluorophenyl)-1H-imidazol-2-yl]carbonyl}-N-(3-fluorophenyl)-4-piperidinamine (E25)
1-{[1-(4-{[(3R,5S)-3,5-dimethyl-1-piperazinyl]methyl}-3-fluorophenyl)-1H-imidazol-2-yl]carbonyl}-N-(2-fluorophenyl)-4-piperidinamine (E26)
N-(3,5-difluorophenyl)-1-{[3-(4-{[(3R,5S)-3,5-dimethyl-1-piperazinyl]methyl}-3-fluorophenyl)-2-pyridinyl]carbonyl}-4-piperidinamine (E27)
1-{[3-(4-{[(3R,5S)-3,5-dimethyl-1-piperazinyl]methyl}-3-fluorophenyl)-2-pyridinyl]carbonyl}-N-[4-fluoro-3-(methyloxy)phenyl]-4-piperidinamine (E28)
3-[(1-{[3-(4-{[(3R,5S)-3,5-dimethyl-1-piperazinyl]methyl}phenyl)-2-pyridinyl]carbonyl}-4-piperidinyl)amino]benzonitrile (E29)
3-[(1-{[3-(4-{[(3R,5S)-3,5-dimethyl-1-piperazinyl]methyl}-3-fluorophenyl)-2-pyridinyl]carbonyl}-4-piperidinyl)amino]benzonitrile (E30)
1-{[3-(4-{[(3R,5S)-3,5-dimethyl-1-piperazinyl]methyl}phenyl)-2-pyridinyl]carbonyl}-N-(2-fluorophenyl)-4-piperidinamine (E31)
1-{[3-(4-{[(3R,5S)-3,5-dimethyl-1-piperazinyl]methyl}phenyl)-2-pyridinyl]carbonyl}-N-[3-(methyloxy)phenyl]-4-piperidinamine (E32)
1-{[3-(4-{[(3R,5S)-3,5-dimethyl-1-piperazinyl]methyl}phenyl)-2-pyridinyl]carbonyl}-N-(3-fluorophenyl)-3-pyrrolidinamine (E33)
(3R,5S)-1-({4-[2-({4-[(3-fluorophenyl)oxy]-1-piperidinyl}carbonyl)-3-pyridinyl]phenyl}methyl)-3,5-dimethylpiperazine (E34)
(3R,5S)-1-({2-fluoro-4-[2-({4-[(3-fluorophenyl)oxy]-1-piperidinyl}carbonyl)-3-pyridinyl]phenyl}methyl)-3,5-dimethylpiperazine (E35)
1-{[3-(4-{[(3R,5S)-3,5-dimethyl-1-piperazinyl]methyl}phenyl)-2-pyridinyl]carbonyl}-N-(4-fluorophenyl)-3-azetidinamine (E36)

The compounds of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

In a further aspect, this invention provides processes for the preparation of a compound of formula (I) or a pharmaceutically acceptable salt or solvate thereof.

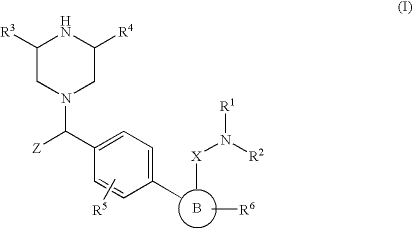

wherein R¹, R², R³, R⁴, R⁵, R⁶, Z and B are as defined in relation to formula (I) and X═CH₂, which process comprises reacting a compound of formula (II),

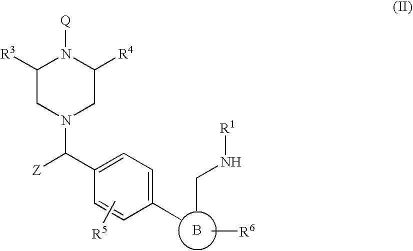

wherein R¹, R³, R⁴, R⁵, R⁶, Z and B are as defined in relation to formula (I) and Q is hydrogen or a nitrogen protecting group such as tert-butyloxycarbonyl (BOC),
with a compound of formula (III),

L-Y—R⁷ (III)

wherein Y and R⁷ are as defined in relation to formula (I) and L is a suitable leaving group such as halogen (e.g. Cl), in the presence of a suitable base, such as triethylamine or pyridine, in a suitable solvent such as tetrahydrofuran or dichloromethane. And thereafter optionally carrying out one or more of the following reactions
1. Converting one compound of formula (I) into another compound of formula (I);
2. Removing any protecting group;
3. Forming a suitable pharmaceutical acceptable salt or solvate of the compound so formed.

In the above process, a compound of formula (I) wherein Y is CO(CH₂)ₙ or CO(CH₂)ₙA may be prepared via an acylation reaction of a compound of formula (II) using appropriate acid chlorides or other activated acid derivatives. A compound of formula (I) wherein Y is SO₂(CH₂)ₙ or SO₂(CH₂)ₙA may be prepared via a sulfonylation reaction of a compound of formula (II) with an appropriate sulfonyl chloride. A compound of formula (I) wherein Y is (CH₂)ₙ or (CH₂)ₙA may be prepared via an alkylation reaction of a compound of formula (II) using an appropriate alkyl halide.

Alternatively, compounds of formula (I) may be prepared by reacting a compound of formula (IV)

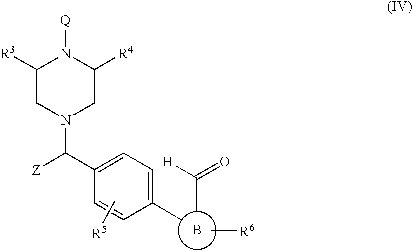

wherein R³, R⁴, R⁵, R⁶, Z and B are as defined in relation to formula (I) and Q is hydrogen or a nitrogen protecting group such as tert-butyloxycarbonyl (BOC), with a compound of formula (V),

HNR¹R² (V)

wherein R¹ and R² are as defined in relation to formula (I) with the proviso that when R² is YR⁷, then Y is (CH₂)ₙ or (CH₂)ₙA; in the presence of a suitable reducing agent, for example sodium borohydride or sodium (triacetoxy)borohydride, in a suitable solvent such as dichloromethane or 1,2-dichloroethane.

Preparation of compounds of formula (II) comprises reacting a compound of formula (IV), wherein R³, R⁴, R⁵, R⁶, Z and B are as defined in relation to formula (I) and Q is hydrogen or a nitrogen protecting group such as tert-butyloxycarbonyl (BOC) with a compound of formula (VI)

H₂NR¹ (VI)

wherein R¹ is as defined in relation to formula (I), in the presence of a suitable reducing agent, for example sodium borohydride, in a suitable solvent such as dichloromethane or 1,2 dichloroethane.

Preparation of compounds of formula (IV) comprises reacting a compound of formula (VII),

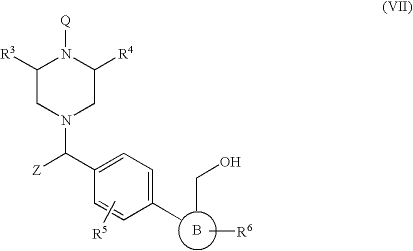

wherein R³, R⁴, R⁵, R⁶, Z and B are as defined in relation to formula (I) and Q is hydrogen or a nitrogen protecting group, with a suitable oxidising agent, for example the Dess-Martin periodinane, in a suitable solvent such as dichloromethane.

Preparation of compounds of formula (VII) comprises reacting a compound of formula (VIII),

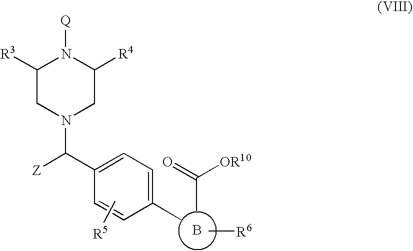

wherein R³, R⁴, R⁵, R⁶, Z and B are as defined in relation to formula (I), R¹⁰ is alkyl, such as methyl or ethyl and Q is hydrogen or a nitrogen protecting group, with a suitable reducing agent, for example lithium aluminium hydride, in a suitable solvent, such as tetrahydrofuran.

Preparation of compounds of formula (VIII) comprises reacting a compound of formula (IX),

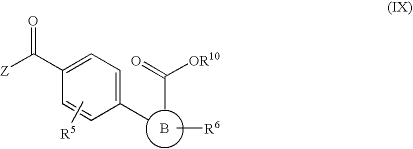

wherein R⁵, R⁶, Z and B are as defined in relation to formula (I) and R¹⁰ is alkyl, such as methyl or ethyl, with an appropriately substituted piperazine (X)

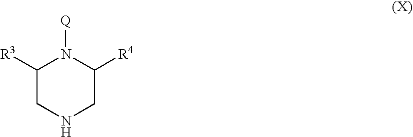

wherein R³ and R⁴ are defined in relation to formula (I) and Q is hydrogen or a nitrogen protecting group such as tert-butyloxycarbonyl, in the presence of a suitable reducing agent, for example sodium (triacetoxy)borohydride, in a suitable solvent such as dichloromethane or 1,2-dichloroethane.

The preparation of compounds of formula (IX) comprises reacting a compound of formula (XI),

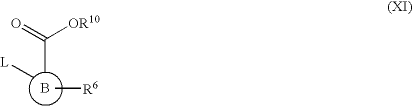

wherein R⁶ is as defined in relation to formula (I), R¹⁰ is alkyl, such as methyl or ethyl, and L is a suitable leaving group such as 1, Br or OSO₂CF₃, with a suitable boronic acid (XII)—

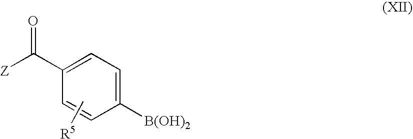

wherein R⁵ and Z are as defined in relation to formula (I)
in the presence of a palladium catalyst, for example Pd(OAc)₂ or Pd(PPh₃)₄ together with a suitable base such as sodium carbonate or potassium carbonate, optionally in the presence of a phase transfer catalyst such as tetrabutylammonium bromide, in a suitable solvent such as a toluene or a mixture of water and 1,2 dimethoxyethane.

Preparation of compounds of formula (XI) wherein L is I or Br, comprises reacting a compound of formula (XIII),

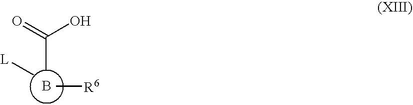

wherein R⁶ and B are as defined in relation to formula (I) and L is I or Br with a suitable alcohol, such as methanol or ethanol, with appropriate acid catalysis, for example sulfuric acid.

According to a further aspect of the invention the preparation of a compound of formula (XI) wherein L is OSO₂CF₃ comprises reacting a compound of formula (XIV),

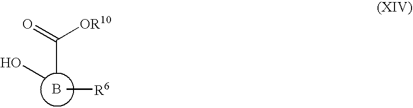

wherein R⁶ and B are as defined in relation to formula (I) and R¹⁰ is alkyl, such as methyl or ethyl, with a suitable trifluoromethanesulfonylating agent such as trifluoromethanesulfonic anhydride or 1,1,1-trifluoro-N-phenyl-N-[(trifluoromethyl) sulfonyl]methanesulfonamide, in the presence of a suitable base such as triethylamine in a suitable solvent such as dichloromethane.

The present invention also provides a further process for the preparation of a compound of formula (I) or a pharmaceutically acceptable salt thereof, wherein X═CO, which process comprises reacting a compound of formula (XV),

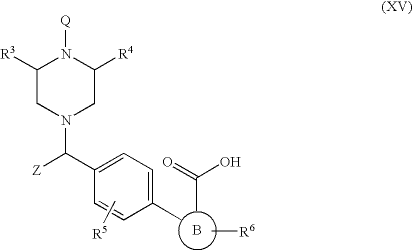

wherein R³, R⁴, R⁵, R⁶, Z and B are as defined in relation to formula (I), and Q is hydrogen or a nitrogen protecting group such as tert-butyloxycarbonyl (BOC) with a compound of formula (V),

HNR¹R² (V)

wherein R¹ and R² are as defined for formula (I) with the proviso that when R² is YR⁷ then Y is (CH₂)ₙ or (CH₂)ₙA, with a suitable coupling reagent such as 1-(3-dimethylaminopropyl)-3-ethyl carbodiimide (EDC) or N-dicyclohexyl carbodiimide (DCC), in a suitable solvent such as dichloromethane and thereafter optionally carrying out one or more of the following reactions
1. Converting one compound of formula (I) into another compound of formula (I);
2. Removing any protecting group;
3. Forming a suitable pharmaceutical acceptable salt or solvate of the compound so formed.

Alternatively, a compound of formula (I) or a pharmaceutically acceptable salt thereof, wherein X═CO, may be prepared by a process which comprises reacting an activated derivative of a compound of formula (XV), such as an acid chloride, with a compound of formula (V), wherein R¹ and R² are as defined for formula (I), using general methods described in J. March, Advanced Organic Chemistry, 4^{th} Edition, J Wiley & Sons, 1992, p. 417-418 and thereafter optionally carrying out one or more of the following reactions:
1. Converting one compound of formula (I) into another compound of formula (I);
2. Removing any protecting group;
3. Forming a suitable pharmaceutical acceptable salt or solvate of the compound so formed.

Preparation of compounds of formula (XV) comprises reacting a compound of formula (VII),

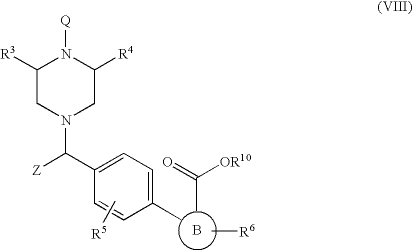

wherein R³, R⁴, R⁵, R⁶, Z and B are as defined in relation to formula (I), R¹⁰ is alkyl, such as methyl or ethyl and Q is hydrogen or a nitrogen protecting group, with a suitable base such sodium hydroxide in a suitable solvent such as a mixture of ethanol or methanol and water.

The present invention also provides a further process for the preparation of a compound of formula (I) or a pharmaceutically acceptable salt thereof, wherein X═CO which process comprises reacting a compound of formula (VII),

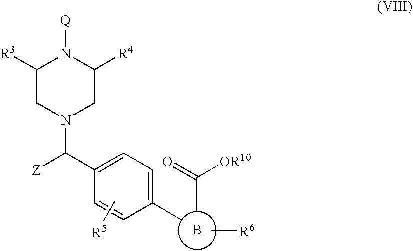

wherein R³, R⁴, R⁵, R⁶, Z and B are as defined in relation to formula (I), R¹⁰ is alkyl, such as methyl or ethyl and Q is hydrogen or a nitrogen protecting group such as tert-butyloxycarbonyl (BOC) with a compound of formula (V),

HNR¹R² (V)

wherein R¹ and R² are as defined for formula (I) with the proviso that when R² is YR⁷ then Y is (CH₂)ₙ or (CH₂)ₙA, in the presence of trimethylaluminium in accordance with the method described in Synth. Commun. 1982, 12, 989-993. The reaction is carried out in a suitable solvent such as toluene. And thereafter optionally carrying out one or more of the following reactions
1. Converting one compound of formula (I) into another compound of formula (I);
2. Removing any protecting group;
3. Forming a suitable pharmaceutical acceptable salt or solvate of the compound so formed.

The present invention also provides a still further process for the preparation of a compound of formula (I) or a pharmaceutically acceptable salt thereof, wherein X═CO, which process comprises reacting a compound of formula (XVI),

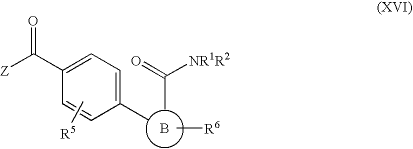

wherein R¹, R², R⁵, R⁶, Z and B are as defined in relation to formula (I), with an appropriately substituted piperazine (X)

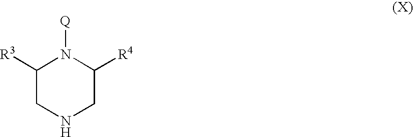

wherein R³ and R⁴ are defined in relation to formula (I) and Q is hydrogen or a nitrogen protecting group such as tert-butyloxycarbonyl, in the presence of a suitable reducing agent, for example sodium (triacetoxy)borohydride, optionally in the presence of a suitable acid catalyst such as acetic acid, in a suitable solvent such as dichloromethane or 1,2-dichloroethane. And thereafter optionally carrying out one or more of the following reactions
1. Converting one compound of formula (I) into another compound of formula (I);
2. Removing any protecting group;
3. Forming a suitable pharmaceutical acceptable salt or solvate of the compound so formed.

Preparation of compounds of formula (XVI) wherein B is 1-imidazolyl, comprises reacting a compound of formula (XVII),

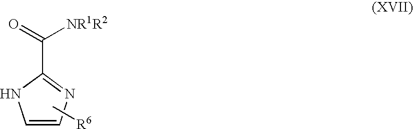

wherein R¹, R², and R⁶ are defined in relation to formula (I) with a suitable boronic acid (XII),

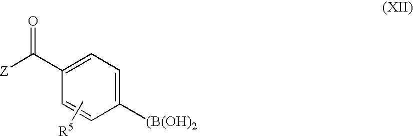

wherein R⁵ and Z are as defined in relation to formula (I), in the presence of a copper catalyst, for example [Cu(OH).TMEDA]₂Cl₂, and oxygen gas, in a suitable solvent such as 1,2-dichloroethane, using a process similar to that described in Organic Letters, 2000, 2, 1233-1236.

Preparation of compounds of formula (XVII) comprises reacting a compound of formula (XVIII),

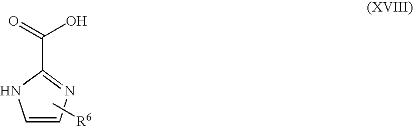

wherein R⁶ is defined in relation to formula (I) with a compound of formula (V),

HNR¹R² (V)

wherein R¹ and R² are as defined for formula (I) with the proviso that when R² is YR⁷ then Y is (CH₂), or (CH₂)ₙA, with a suitable coupling reagent such as 1-(3-dimethylaminopropyl)-3-ethyl carbodiimide (EDC) or N-dicyclohexyl carbodiimide (DCC), in a suitable solvent such as dichloromethane.

According to a further aspect of the invention the preparation of compounds of formula (XVII) comprises reacting a compound of formula (XIX),

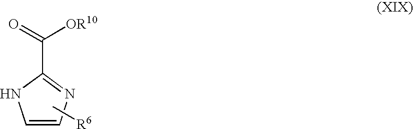

wherein R⁶ is defined in relation to formula (I), R¹⁰ is alkyl, such as methyl or ethyl with a compound of formula (V),

HNR¹R² (V)

wherein R¹ and R² are as defined for formula (I) with the proviso that when R² is YR⁷ then Y is (CH₂)ₙ or (CH₂)ₙA, in the presence of trimethylaluminium in accordance with the method described in Synth. Commun. 1982, 12, 989-993. The reaction is carried out in a suitable solvent such as toluene.

According to a further aspect of the present invention, the preparation of compounds of formula (XVI) wherein B is pyridyl, comprises reacting a compound of formula (XX),

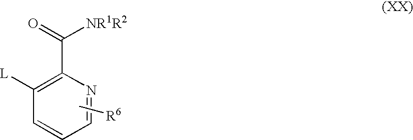

wherein R¹, R², and R⁶ are defined in relation to formula (I) and L is a suitable leaving group such as 1, Br or OSO₂CF₃ with a suitable boronic acid (XII),

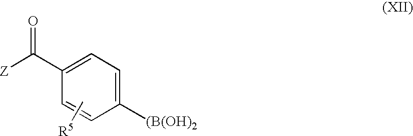

wherein R⁵ and Z are as defined in relation to formula (I), in the presence of a palladium catalyst, for example Pd(OAc)₂ or Pd(PPh₃)₄ together with a suitable base such as sodium carbonate or potassium carbonate, optionally in the presence of a phase transfer catalyst such as tetrabutylammonium bromide, in a suitable solvent such as a toluene or a mixture of water and 1,2 dimethoxyethane.

Preparation of compounds of formula (XX), wherein L is I or Br, comprises reacting a compound of formula (XXI),

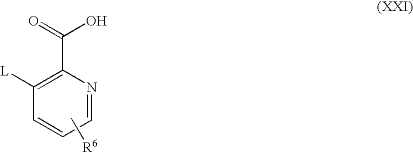

wherein R⁶ is defined in relation to formula (I), with a compound of formula (V),

HNR¹R² (V)

wherein R¹ and R² are as defined for formula (I) with the proviso that when R² is YR⁷ then Y is (CH₂)ₙ or (CH₂)ₙA, with a suitable coupling reagent such as 1-(3-dimethylaminopropyl)-3-ethyl carbodiimide (EDC) or N-dicyclohexyl carbodiimide (DCC), in a suitable solvent such as dichloromethane.

According to a further aspect of the invention the preparation of a compound of formula (XX) wherein L is OSO₂CF₃ comprises reacting a compound of formula (XXII),

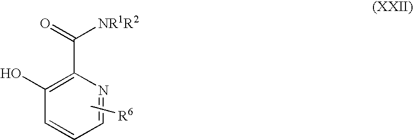

wherein R¹, R², and R⁶ are defined in relation to formula (I), with a suitable trifluoromethanesulfonylating agent such as trifluoromethanesulfonic anhydride or 1,1,1-trifluoro-N-phenyl-N-[(trifluoromethyl) sulfonyl]methanesulfonamide in the presence of a suitable base such as triethylamine in a suitable solvent such as dichloromethane.

The preparation of a compound of formula (XXII) comprises reaction a compound of formula (XXIII),

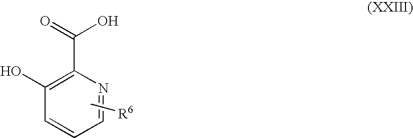

wherein R⁶ is defined in relation to formula (I), with a compound of formula (V),

HNR¹, R² (V)

wherein R¹ and R² are as defined for formula (I) with the proviso that when R² is YR⁷ then Y is (CH₂)ₙ or (CH₂)ₙA, in the presence of a suitable activating agent, such as isobutylchloroformate, and a suitable base, such as triethylamine, in a suitable solvent such as tetrahydrofuran; followed by treatment with an aqueous base, for example aqueous sodium hydroxide.

The present invention also provides a further process for the preparation of a compound of formula (I) or a pharmaceutically acceptable salt thereof, wherein X═SO₂ which process comprises reacting a compound of formula (XXIV),

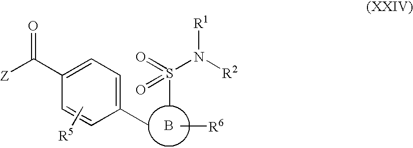

wherein R¹, R², R⁵, R⁶, Z and B are as defined in relation to formula (I) with an appropriately substituted piperazine (X)

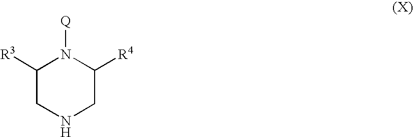

wherein R³ and R⁴ are defined in relation to formula (I) and Q is hydrogen or a nitrogen protecting group such as tert-butyloxycarbonyl, in the presence of a suitable reducing agent, for example sodium (triacetoxy)borohydride, in a suitable solvent such as dichloromethane or 1,2-dichloroethane and thereafter optionally carrying out one or more of the following reactions
1. Converting one compound of formula (I) into another compound of formula (I);
2. Removing any protecting group;
3. Forming a suitable pharmaceutical acceptable salt or solvate of the compound so formed.

The preparation of compounds of formula (XXIV) comprises reacting a compound of formula (XII),

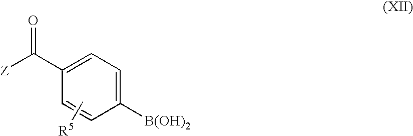

wherein R⁵ and Z are as defined as in relation to formula (I) with a compound of formula (XXV),

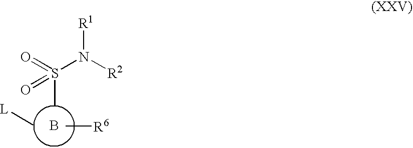

where R¹, R², R⁶ and B are as defined in relation to formula (I) and L is a suitable leaving group, such as I or Br, in the presence of a palladium catalyst, for example Pd(OAc)₂, together with a suitable base such as sodium carbonate optionally in the presence of a phase transfer catalyst such as tetrabutylammonium bromide in a suitable solvent, such as a mixture of water and 1,2 dimethoxyethane.

Preparation of compounds of formula (XXV) comprises reacting a compound of formula (XXVI),

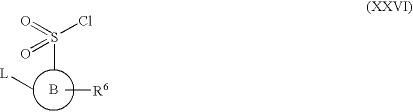

wherein R⁶ is as defined in relation to formula (I) and L is a leaving group, such as Br or I, with a compound of formula (V),

HNR¹R² (V)

wherein R¹ and R² are as defined in relation to formula (I) with the proviso that when R² is YR⁷ then Y is (CH₂)ₙ or (CH₂)ₙA, in the presence of a base such as triethylamine or pyridine in a suitable solvent such as tetrahydrofuran or dichloromethane.

Alternatively, a compound of formula (I) or a pharmaceutically acceptable salt thereof, wherein X═SO₂ and R¹, R² and the nitrogen to which they are attached form a piperidine ring substituted by WR⁷, wherein W is NH or N—C₍₁₋₄₎alkyl, may be prepared by a process which comprises reacting a compound of formula (XXVII)

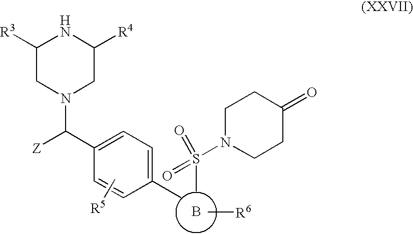

wherein R³, R⁴, R⁵, R⁶, Z and B are defined in relation to formula (I) with a compound of formula (XXVIII)

HWR⁷ (XXVIII)

wherein W is NH or N—C₍₁₋₄₎alkyl and R⁷ are defined in relation to formula (I), in the presence of a suitable reducing agent such as sodium (triacetoxy)borohydride with a suitable acid catalyst such as acetic acid, in a suitable solvent such as dichloromethane or 1,2-dichloroethane. And thereafter optionally carrying out one or more of the following reactions
1. Converting one compound of formula (I) into another compound of formula (I);
2. Removing any protecting group;
3. Forming a suitable pharmaceutical acceptable salt or solvate of the compound so formed.

Preparation of compounds of formula (XXVII) comprises reaction a compound of formula (XXIX)

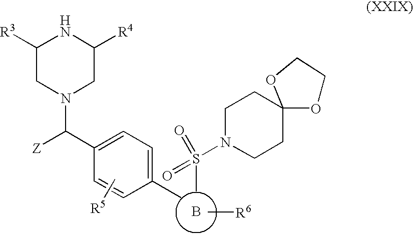

wherein R³, R⁴, R⁵, R⁶, Z and B are defined in relation to formula (I) with a suitable acid such as hydrochloric acid, in a suitable solvent such as acetone.

Preparation of compounds of formula (XXIX) comprises reaction a compound of formula (XXX)

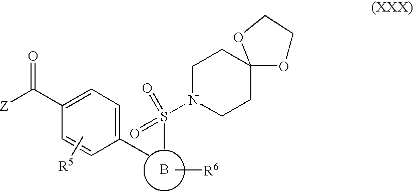

wherein R⁵, R⁶, Z and B are defined in relation to formula (I) with an appropriately substituted piperazine (X)

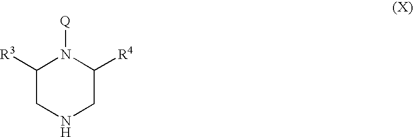

wherein R³ and R⁴ are defined in relation to formula (I) and Q is hydrogen or a nitrogen protecting group such as tert-butyloxycarbonyl, in the presence of a suitable reducing agent, e.g. sodium (triacetoxy)borohydride, in a suitable solvent such as dichloromethane or 1,2-dichloroethane.

Preparation of compounds of formula (XXX) comprises reaction a compound of formula (XXXI)

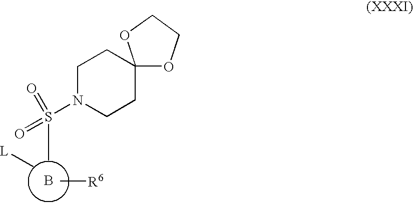

wherein R⁶ and B are as defined in relation to formula (I) and L is a suitable leaving group such as I or Br, with a compound of formula (XII),

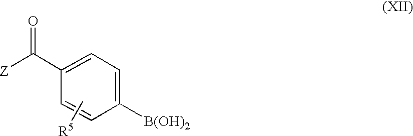

wherein R⁵ and Z are as defined as in relation to formula (I), in the presence of a palladium catalyst, e.g. Pd(PPh₃)₄, together with a suitable base such as sodium carbonate, in a suitable solvent, such as a mixture of water and 1,2 dimethoxyethane.

Preparation of compounds of formula (XXXI), wherein L=I, comprises reaction a compound of formula (XXXII)

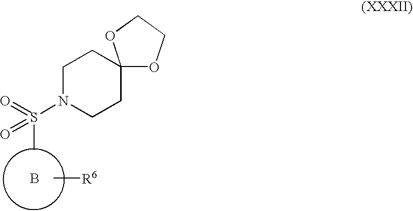

wherein R⁶ and B are defined in relation to formula (I) with a suitable base, e.g. lithium diisopropylamide, followed by treatment with iodine in a suitable solvent such as tetrahydrofuran.

Preparation of compounds of formula (XXXII) comprises reacting a compound of formula (XXXIII),

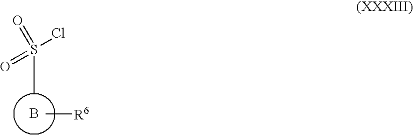

wherein R⁶ is as defined in relation to formula (I) with a compound of formula (XXXIV),

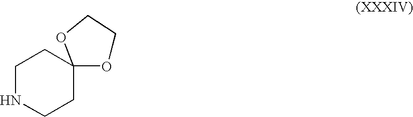

in the presence of a suitable base, e.g. triethylamine or pyridine, in a suitable solvent such as dichloromethane.

It will be appreciated by those skilled in the art that it may be necessary to protect certain reactive substituents during some of the above procedures. Standard protection and deprotection techniques, such as those described in Greene T. W. Protective groups in organic synthesis, New York, Wiley (1981), can be used. For example, primary amines can be protected as phthalimide, benzyl, tert-butyloxycarbonyl, benzyloxycarbonyl or trityl derivatives. Carboxylic acid groups can be protected as esters. Aldehyde or ketone groups can be protected as acetals, ketals, thioacetals or thioketals. Deprotection of such groups is achieved using conventional procedures well known in the art. For example, protecting groups such as tert-butyloxycarbonyl may be removed using an acid such as hydrochloric or trifluororoacetic acid in a suitable solvent such as dichloromethane, diethylether, isopropanol or mixtures thereof.

Pharmaceutically acceptable salts may be prepared conventionally by reaction with the appropriate acid or acid derivative.

The present invention also provides compounds of formula (II), (XV), (XXIV), (IV), (VII), (XVI) or (XXVII) as shown above in which R¹, R², R³, R⁴, R⁵, R⁶, Z and B are as defined in claim 1, R¹⁰ is C₁₋₄alkyl, and Q is hydrogen or a nitrogen protecting group. Those compounds are useful as intermediates in the preparation of compounds of the present invention. The invention further provides compounds (VII), (XXIX) and (XXX) as shown above in which R³, R⁴, R⁵, R⁶, Z and B are as defined in claim 1, and Q is hydrogen or a nitrogen protecting group. The invention still further provides compounds (IX), (XXXI) and (XXXII) as shown above in which R⁵, R⁶, Z and B are as defined in claim 1, R¹⁰ is C₁₋₄alkyl, and Q is hydrogen or a nitrogen protecting group. Those compounds are useful as intermediates in the preparation of compounds of the present invention.

The potencies and efficacies of the compounds of this invention for GPR³⁸ can be determined by FLIPR assay performed on the human cloned receptor as described herein. It has been found, using the FLIPR functional assay, that compounds of formula (I) appear to be partial or full agonists of the GPR38 receptor.

The potencies and intrinsic activities of the compounds of this invention can also be determined according to the [35S]GTPγS functional assay which is described herein. It has been found, using the [35S]GTPγS functional assay, that compounds of formula (I) appear to be partial or full agonists of the GPR38 receptor.

Compounds of formula (I) and their pharmaceutically acceptable salts are therefore of use in the treatment of conditions or disorders which are mediated by compounds acting at the GPR38 receptor. In particular the compounds of formula (I) and their pharmaceutically acceptable salts are of use in the treatment of certain gastrointestinal disorders such as gastroesophageal reflux disorders, functional dyspepsia, irritable bowel syndrome, constipation, intestinal pseudo-obstruction, paralytic ileus following surgery or other manipulation, emesis, gastric stasis or hypomotility caused by various diseases such as diabetes and/or by the administration of other drugs, Crohn's disease, colitis, cachexia associated with advanced diseases such as cancer and/or the treatment thereof, and other disorders such as incontinence.

It is to be understood that “treatment” as used herein includes prophylaxis as well as alleviation of established symptoms.

Thus the invention also provides a compound of formula (I) or a pharmaceutically acceptable salt thereof, for use as a therapeutic substance, in particular in the treatment of the conditions/disorders which can be mediated via the GPR38 receptor. In particular the invention provides a compound of formula (I) or a pharmaceutically acceptable salt thereof for use as a therapeutic substance in the treatment of gastrointestinal disorders such as gastro-esophageal reflux disease, functional dyspepsia, irritable bowel syndrome, constipation, intestinal pseudo-obstruction, paralytic ileus following surgery or other manipulation, emesis, gastric stasis or hypomotility caused by various diseases such as diabetes and/or by the administration of other drugs, Crohn's disease, colitis, cachexia associated with advanced diseases such as cancer and/or the treatment thereof, and other disorders such as incontinence.

The invention further provides a method of treatment of conditions or disorders in mammals including humans which can be mediated via the GPR38 receptor, which comprises administering to the sufferer a therapeutically safe and effective amount of a compound of formula (I) or a pharmaceutically acceptable salt thereof.

In another aspect, the invention provides for the use of a compound of formula (I) or a pharmaceutically acceptable salt thereof in the manufacture of a medicament for use in the treatment of the conditions or disorders mediated via the GPR38 receptor

In order to use the compounds of formula (I) in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salt thereof and a sterile vehicle. Formulations for injection may be presented in unit dosage form e.g. in ampoules or in multi-dose, utilising a compound of the invention or pharmaceutically acceptable salt thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unitary dose device or alternatively as a powder mix with a suitable carrier for administration using a suitable delivery device. Thus compounds of formula (I) may be formulated for oral, buccal, parenteral, topical (including ophthalmic and nasal), depot or rectal administration or in a form suitable for administration by inhalation or insufflation (either through the mouth or nose).

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, for example from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 200 mg, and such unit doses may be administered more than once a day, for example two or three times a day. Such therapy may extend for a number of weeks or months.

The compounds of the present invention may be used in combination preparations. For example, the compounds of the invention may be used in combination with one or more compounds with activity in reducing gastric acid; one or more compounds with activity in reducing gastro-esophageal reflux; one or more compounds with activity in reducing esophago-gastric irritancy or inflammation, especially when used to alleviate erosive or non-erosive esophagitis; one or more compounds with analgesic activity; and/or one or more compounds with mixed activity on motility and pain.

Examples of compounds with activity in reducing gastric acid include H2 receptor antagonists, acid pump antagonists and proton pump inhibitors. Examples of compounds with activity in reducing gastro-esophageal reflux include agonists at GABA-B. Examples of compounds with analgesic activity include compounds active at Neurokinin receptors (NK1, 2, 3), TRPV1 and sodium-channels. Examples of compounds with mixed activity on motility and pain include CRF2 antagonists, 5-HT3 antagonists or octreotide or other molecules active at sst2 receptors.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

### Abbreviations

HCl—hydrochloric acid, H₂SO₄—sulfuric acid,
NaHCO₃— sodium hydrogen carbonate, Na₂SO₄—sodium sulfate,
NaOH—sodium hydroxide, DCM—dichloromethane,
DMF—N,N-dimethylformamide, THF—tetrahydrofuran
MeOH—methanol, EtOAc—ethyl acetate
Na₂CO₃—sodium carbonate, MgSO₄—magnesium sulphate
1,2-DME—1,2-dimethoxyethane, NH₃—ammonia
TMEDA—N,N,N′N′-tetramethylethylenediamine
dppf—1,1′-bis(diphenylphosphino)ferrocene

### Description 1

### 3-Iodo-2-pyridinecarboxylic acid (D1)

To a stirred solution of 2,2,6,6-tetramethylpiperidine (20 ml, 0.122 mol) in dry THF (100 ml) at −78° C., under argon was added n-butyllithium (52 ml, 0.163 mol, 2.5M solution in hexanes) dropwise, followed 15 min later by a solution of 2-pyridinecarboxylic acid (5.0 g, 0.0407 mol) in dry THF (30 ml). After 10 min at −78° C., the reaction mixture was warmed to 0° C. for 30 min. and then transferred to a solution of iodine (30.9 g, 0.243 mol) in dry THF (70 ml) at 0° C., under argon. After 15 min at 0° C. the reaction mixture was warmed to 25° C. and stirred for 1 h. After this period water (80 ml) was added and the reaction mixture concentrated in vacuo. The residue was re-dissolved in water (100 ml) and washed with EtOAc (100 ml). The aqueous layer was separated, concentrated in vacuo and the resulting residue triturated with diethyl ether. The solid material was filtered and dried in vacuo before being re-dissolved in MeOH (200 ml). To this solution was added Amberlyte IR-120 ion-exchange resin (100 g) and the reaction mixture stirred at 25° C. for 2 h. After this period the resin was filtered off and the solvents concentrated in vacuo to afford the title compound (4.15 g, 41%). δH (DMSO-d₆, 250 MHz) 6.79 (1H, bs) 7.28 (1H, dd), 8.37 (1H, dd), 8.58 (1H, dd). MS (ES): C₆H₄₁NO₂ requires 249. found (M-H⁺) 248.

### Description 2

### Methyl 3-iodo-2-pyridinecarboxylate (D2)

A mixture of D1 (3.0 g, 0.012 mol) and c.H₂SO₄ (2 ml) in MeOH (100 ml) was heated at 65° C. for 18 h. After this period, solvents were evaporated in vacuo and the residue basified with solid NaHCO₃. The residue was then extracted with EtOAc (3×100 ml). The organic layer was separated, dried (Na₂SO₄) and concentrated in vacuo to afford the title compound (2.2 g, 70%). δH(CDCl₃, 250 MHz) 4.01 (3H, s), 7.13 (1H, dd), 8.29 (1H, dd), 8.64 (1H, dd). MS (ES): C₇H₆₁NO₂ requires 263. found 264 (MH⁺)

### Description 3

### Methyl 3-(4-formylphenyl)-2-pyridinecarboxylate (D3)

A suspension of D2 (0.2 g, 0.76 mmol), (4-formylphenyl)boronic acid (0.114 g, 0.76 mmol), palladium (II) acetate (17 mg, 0.076 mmol), sodium carbonate (80 mg, 0.76 mmol) and tetrabutylammonium bromide (244 mg, 0.76 mmol) in (1:1) water/1,2-dimethoxyethane (6 ml) were sonicated for 5 min. and then the reaction heated in an Emrys™ Optimizer EXP microwave reactor (120° C. for 5 min). The reaction mixture was then diluted with EtOAc (20 ml) and water (8 ml). The organic layer was separated, washed with brine, dried (Na₂SO₄) and concentrated in vacuo. The crude material was purified by column chromatography on silica eluting with a 0-100% EtOAc/40-60 petroleum ether gradient to afford the title compound (87 mg, 50%), δH (CDCl₃, 250 MHz) 3.81 (3H, s), 7.49-7.57 (3H, m), 7.77 (1H, dd), 7.96 (2H, dd), 8.75 (1H, dd), 10.09 (1H, s). MS (ES): C₁₄H₁₁NO₃ requires 241. found 242 (MH⁺)

### Description 3 (Alternative Procedure)

### Methyl 3-(4-formylphenyl)-2-pyridinecarboxylate (D3)

A suspension of D7 (8.26 g, 30 mmol), 4-formylphenyl boronic acid (5.65 g, 38 mmol) and potassium carbonate (5.21 g, 38 mmol) in dry toluene (150 ml) was stirred at 25° C. under argon and tetrakistriphenylphosphine palladium(0) (0.83 g, 0.72 mmol) was added. The mixture was heated at reflux for 16 h and work-up using a method similar to that of Description 3 afforded the title compound as a yellow solid (5 g).

### Description 4

### Methyl-3-(4-{[(3R,5S)-3,5-dimethyl-1-piperazinyl]methyl}phenyl)-2-pyridine carboxylate (D4)

To a solution of D3 (260 mg, 1.08 mmol) in dry DCM (30 ml) was added (2R,6S)-dimethylpiperazine (185 mg, 1.62 mmol) at 0° C. under argon. The reaction mixture was then warmed to 25° C. and stirred for 1 h. After this period, sodium (triacetoxy)borohydride (343 mg, 1.62 mmol) was added portionwise and the reaction mixture stirred at 25° C. under argon for 18 h. The reaction was then quenched with saturated NaHCO₃ solution (50 ml) and extracted with DCM (3×50 ml). The organic layers were combined, washed with water, dried (Na₂SO₄) and concentrated in vacuo. The crude oil was purified by column chromatography on silica eluting with a 0-10% [(9:1)MeOH:ammonia]/EtOAc gradient to afford the title compound (213 mg, 58%). δH (CDCl₃, 250 MHz) 1.04 (6H, d), 1.64 (2H, t), 2.79 (2H, m), 2.89-2.99 (2H, m), 3.54 (2H, s), 3.79 (3H, s), 7.28-7.50 (5H, m), 7.77 (1H, dd), 8.66 (1H, dd). MS (ES): C₂₀H₂₅N₃O₂ requires 339. found 340 (MH⁺)

### Description 5

### 3-(4{[(3R,5S)-3,5-Dimethyl-1-piperazinyl]methyl}phenyl)-2-pyridinecarboxylic acid (D5)

A solution of D4 (72 mg, 0.212 mmol) and 2M NaOH (3 ml) in dioxane (3 ml) was stirred at 25° C. for 7 h. After this period the reaction mixture was acidified to pH6 with 2M HCl and the solvents concentrated in vacuo to afford the title compound isolated as a di-HCl salt (69 mg, 100%). δH (DMSO-d₆, 400 MHz) 1.23 (6H, d), 2.17 (2H, t), 2.86 (2H, m), 3.16 (2H, m), 3.58 (2H, d), 4.15 (1H, bs), 7.37 (2H, d), 7.46 (3H, m), 7.82 (1H, dd), 8.51 (1H, dd). MS (ES): C₁₉H₂₃N₃O₂ requires 325. found 324 (M-H)⁺

### Description 6

### Methyl 3-hydroxy-2-pyridine carboxylate (D6)

A mixture of 3-hydroxy-2-pyridine carboxylic acid (25 g, 180 mmol) in MeOH (200 ml) containing c.H₂SO₄ (50 ml) was heated at reflux for 2 days. The mixture was concentrated in vacuo, diluted with water (250 ml) and neutralised with Na₂CO₃ to pH6. The mixture was extracted with DCM (3×200 ml) which was washed with water then dried (Na₂SO₄) and concentrated in vacuo to afford the title compound as a white solid (21.5 g). δH (CDCl₃, 250 MHz) 4.07 (3H, s), 7.41 (2H, m), 8.29 (1H, dd) 10.64 (1H, s). MS (ES): MH⁺ 154.

### Description 7

### Methyl 3-trifluoromethanesulfonyloxy-2-pyridine carboxylate (D7)

D6 (15 g, 98 mmol) in DCM (196 ml) was cooled (ice-bath) and treated with triethylamine (14.9 ml) followed by portionwise addition of trifluoromethanesulfonic anhydride (18 ml, 108 mmol). The mixture was stirred at 25° C. for 2 h, washed with water and saturated NaHCO₃ solution, then dried (Na₂SO₄). The solvent was removed in vacuo to give the title compound as an orange oil (28 g). δH (CDCl₃, 250 MHz) 4.05 (3H, s), 7.65 (1H, dd), 7.75 (1H, m), 8.77 (1H, dd).

### Description 8

### Methyl-3-(4-{[(3R,5S)-3,5-dimethyl-4-tert-butoxycarbonyl-1-piperazinyl]methyl}phenyl)-2-pyridine carboxylate (D8)

A solution of D3 (2.75 g, 11.41 mmol) and (2R,6S)-dimethyl-1-tert-butoxycarbonylpiperazine (2.44 g, 11.41 mmol) (prepared by a method similar to that described in H. Ishida et al, PCT Int. Appl., WO 2003063874, for the preparation of (2R,6R)-dimethyl-1-tert-butoxycarbonylpiperazine) in 1,2-dichloroethane (50 ml) was stirred at 25° C. for 24 h. Sodium (triacetoxy)borohydride (3.63 g, 17.12 mmol) was added and the mixture stirred overnight. The mixture was diluted with DCM, washed with saturated NaHCO₃ solution and water, then dried (Na₂SO₄). Concentration in vacuo followed by chromatography on silica using a 0-50% EtOAc/40-60 petroleum ether gradient afforded the title compound as a white foam (2.36 g). δH (CDCl₃, 250 MHz) 1.31 (6H, d), 1.47 (9H, s), 2.17 (2H, dd), 2.64 (2H, d), 3.54 (2H, s), 3.78 (3H, s), 4.11 (2H, m), 7.30 (2H, d), 7.46 (3H, m), 7.47 (1H, dd), 8.66 (1H, dd). MS (ES): MH⁺ 440.

### Description 9

### 3-(4-{[(3R,5S)-3,5-Dimethyl-4-tert-butoxycarbonyl-1-piperazinyl]methyl}phenyl)-2-pyridine carboxylic acid (D9)

The ester D8 (2.36 g, 5.38 mmol) in dioxane (25 ml) was treated with a solution of lithium hydroxide (0.451 g, 10.75 mmol) in water (25 ml) and stirred at 25° C. for 3 h then concentrated in vacuo. The residue was acidified to pH5 with 2M HCl and extracted with EtOAc (×3). The combined organics were dried and concentrated in vacuo to give the title compound as a white solid (2.28 g). δH (CDCl₃, 250 MHz) 1.32 (6H, d), 1.47 (9H, s), 2.18 (2H, dd), 2.70 (2H, d), 3.57 (2H, s), 4.11 (2H, m), 7.32 (2H, d), 7.43 (2H, m), 7.58 (1H, dd), 7.81 (1H, dd), 8.61 (1H, dd). MS (ES): (M-H⁺) 424.

### Description 10

### 1-tert-Butoxycarbonyl-4-[(4-fluorophenyl)amino]piperidine (D10)

A solution of 1-(tert-butoxycarbonyl)piperidin-4-one (1 g, 5 mmol), 4-fluoroaniline (0.56 g, 5 mmol) and acetic acid (0.286 ml, 5 mmol) in 1,2-dichloroethane (30 ml) was stirred at room temperature for 24 h. Sodium (triacetoxy)borohydride (1.48 g, 7 mmol) was then added and stirring continued for 24 h. The reaction mixture was washed with water, dried (MgSO₄) and then concentrated in vacuo to give the title compound as a solid (1.44 g). δH (CDCl₃, 250 MHz) 1.30 (2H, m), 1.46 (9H, s), 2.02 (2H, m), 2.91 (2H, m), 3.35 (1H, m), 4.04 (2H, m), 6.54 (2H, dd), 6.88 (2H, t).

### Description 11

### 4-[(4-Fluorophenyl)amino]piperidine (D11)

A solution of D10 (1.44 g) in 2M HCl (5 ml) and 1,4-dioxane (20 ml) was heated at 60° C. for 24 h. On cooling, the solution was diluted with water, basified with 2M NaOH solution and extracted with EtOAc (×3). The combined organics were dried (MgSO₄) and concentrated in vacuo to give the title compound as a yellow oil (0.71 g). δH (CDCl₃, 250 MHz) 1.29 (2H, m), 2.05 (2H, m), 2.70 (2H, m), 3.20 (1H, m), 3.30 (2H, m), 6.54 (2H, dd), 6.88 (2H, t).

The following intermediates were prepared from the appropriate aniline using the methods outlined in Descriptions 10 and 11.
4-[(3-Fluorophenyl)amino]piperidine (D12)
4-[(2-Fluorophenyl)amino]piperidine (D13)
4-[(3,4-Difluorophenyl)amino]piperidine (D14)

### Description 15

### (3R,5S)-3,5-Dimethyl-1-({4-[2-({4-[(4-fluorophenyl)amino]piperidin-1-yl}carbonyl)pyridin-3-yl]phenyl}methyl)-4-tert-butoxycarbonyl-piperazine (D15)

Acid D9 (1.0 g, 2.35 mmol), 1-(3-dimethylaminopropyl)-3-ethylcarbodiimide hydrochloride (0.677 g, 3.53 mmol) and 1-hydroxybenzotriazole (0.476 g, 3.53 mmol) were stirred in dry DMF (20 ml) for 2 hours. D11 (0.456 g, 2.35 mmol) was added and the mixture stirred at 25° C. overnight, then the solvent removed in vacuo. The residue was taken up in DCM and washed with saturated NaHCO₃ solution and brine. The DCM layer was dried and concentrated to produce crude product as a brown oil (1.54 g). The product was purified by column chromatography to produce product as a pale yellow foam (0.92 g). δH (CDCl₃, 250 MHz): 0.74 (1H, m), 1.19 (1H, m), 1.30 (6H, dd), 1.47 (9H, s), 1.62 (1H, br), 1.96 (1H, br), 2.19 (2H, m), 2.64 (2H, d), 2.70-2.90 (2H, br m), 3.15-3.40 (2H, br m), 3.55 (2H, s), 4.00-4.20 (2H, m), 4.50 (1H, m), 6.44 (2H, m), 6.83 (2H, t), 7.33-7.55 (5H, m), 7.78 (1H, dd), 8.61 (1H, dd).

### Description 16

### [3-(4-{[(3R,5S)-3,5-Dimethyl-1-piperazinyl]methyl}phenyl)-2-pyridinyl]methanol (D16)

To a stirred solution of D4 (0.1 g, 0.29 mmol) in dry THF (10 ml) at −78° C. under argon was added lithium aluminium hydride (0.35 ml, 0.35 mmol, 1M solution in THF) dropwise. The reaction mixture stirred at −78° C. for 1 h, before a further equivalent of lithium aluminium hydride was added (0.29 ml, 0.29 mmol) and the reaction mixture warmed to 25° C. overnight. After this period the reaction was quenched with water (0.3 ml), 2M NaOH (0.6 ml) and water (0.4 ml), stirred for 30 min. and then filtered through Celite. The solid residue was extracted with EtOAc (4×10 ml), DCM (2×10 ml), the combined organic layers dried (Na₂SO₄) and then concentrated in vacuo to afford the title compound (89 mg). δH (CDCl₃, 250 MHz) 0.97 (6H, d), 1.58 (2H, t), 2.72 (2H, dd), 2.82-2.96 (2H, m), 3.47 (2H, s), 4.60 (2H, s), 6.91-7.27 (3H, m), 7.33 (2H, d), 7.53 (1H, dd), 8.50 (1H, dd). MS (ES): MH⁺ 312.

### Description 17

### 3-(4{[(3R,5S)-3,5-dimethyl-1-piperazinyl]methyl}phenyl)-2-pyridine carbaldehyde (D17)

To a stirred solution of D16 (90 mg, 0.289 mmol) in dry DCM (5 ml) was added a suspension of 1,1,1-tris(acetyloxy)-1,1-dihydro-1,2-benziodoxol-3-(1H)-one (147 mg, 0.347 mmol) in dry DCM (3 ml) and the reaction mixture stirred at −78° C. for 45 min before warming to 25° C. for 2 h. After this period, further 1,1,1-tris(acetyloxy)-1,1-dihydro-1,2-benziodoxol-3-(1H)-one (31 mg, 0.073 mmol) was added and the reaction mixture stirred at 25° C. overnight. The reaction was then quenched with a 1:1 mixture of sodium thiosulphate solution (10% w/v) and saturated NaHCO₃ solution (30 ml) then extracted with DCM (3×50 ml). The combined organic layers were separated, dried (Na₂SO₄) and concentrated in vacuo to afford the title compound (75 mg). δH (CDCl₃, 250 MHz) 1.03 (6H, d), 1.69 (2H, t), 2.71 (2H, dd), 2.89-3.01 (2H, m), 3.49 (2H, s), 7.27-7.38 (4H, m), 7.45-7.54 (1H, m), 7.74 (1H, dd), 8.76 (1H, dd), 10.0 (1H, s). MS (ES): MH⁺ 310.

### Description 18

### {[3-(4{[(3R,5S)-3,5-Dimethyl-1-piperazinyl]methyl}phenyl)-2-pyridinyl]methyl}methylamine (D18)

Methylamine (0.15 ml, 0.243 mmol, 2M solution in THF) was added to a stirred solution of D17 (75 mg, 0.243 mmol) in dry MeOH (10 ml) at 25° C. under argon and the reaction mixture stirred at 25° C. for 18 h. Another 0.3 ml (0.6 mmol) of methylamine solution was then added and the reaction left for a further 6 h. After this period, sodium borohydride (9 mg, 0.243 mmol) was added and reaction stirred for 1 h. The reaction mixture was then partitioned between 2M NaOH (10 ml) and DCM (10 ml) and the organic layer separated. The aqueous layer was then re-extracted with DCM (2×10 ml) and the combined organic layers dried (Na₂SO₄) and concentrated in vacuo to afford the title compound (75 mg). δH (CDCl₃, 250 MHz) 1.05 (6H, d), 1.65 (2H, t), 2.39 (3H, s), 2.75-2.85 (2H, m), 2.90-3.00 (2H, m), 3.54 (2H, s), 3.81 (2H, s), 7.20-7.32 (3H, m), 7.39 (2H, dd), 7.52-7.62 (1H, m), 8.57 (1H, m). MS (ES): MH⁺ 325.

### Description 19

### Methyl 5-(4-formylphenyl)-2-methyl-1,3-thiazole-4-carboxylate (D19)

To a solution of 4-[bis(ethyloxy)methyl]benzaldehyde (5 g, 24 mmol) and methyl dichloroacetate (2.49 ml, 24 mmol) in dry diethyl ether (25 ml) at 5° C., under argon was added sodium methoxide (1.95 g, 36 mmol) portionwise, keeping the temperature below 8° C. The reaction mixture was stirred at this temperature for 1 h and then allowed to warm to 25° C. and heated at reflux for a further 2 h. The reaction mixture was cooled, diluted with ether (10 ml) and washed with water (2×10 ml). The organic layer was separated, washed with brine, dried (Na₂SO₄) and then concentrated in vacuo. The crude material was purified by column chromatography on silica eluting with a 0-50% EtOAc/40-60 petroleum ether gradient to afford the intermediate methyl 3-{4-[bis(ethyloxy)methyl]phenyl}-3-chloro-2-oxopropanoate (5.15 g, 68%). This compound (1 g, 3.18 mmol) was dissolved in ethanol (5 ml) and added dropwise over 30 min. to a refluxing solution of thioacetamide (0.238 g, 3.18 mmol) in ethanol (10 ml) under argon. After addition, the reaction mixture was heated at reflux overnight. The solvent was then evaporated and residues partitioned between EtOAc (20 ml) and water (20 ml). The organic layer was separated and the aqueous layer re-extracted with EtOAc (2×20 ml), washed with saturated NaHCO₃ solution (10 ml), brine (10 ml), dried (Na₂SO₄) and then concentrated in vacuo. The crude material was purified by column chromatography on silica eluting with a 0-50% EtOAc/40-60 petroleum ether gradient to afford the title compound (0.264 g). δH (CDCl₃, 250 MHz) 2.78 (3H, s), 3.86 (3H, s), 7.66 (2H, d), 7.93 (2H, d), 10.6 (1H, s). MS (ES): MH⁺ 262.

### Description 20

### Methyl-5-(4-{[(3R,5S)-3,5-dimethyl-1-piperazinyl]methyl}phenyl)-2-methyl-1,3-thiazole-4-carboxylate (D20)

To a solution of D19 (748 mg, 2.86 mmol) in dry DCM (10 ml) was added (2R,6S)-dimethylpiperazine (654 mg, 5.73 mmol) at 0° C. under argon. The reaction mixture was warmed to 25° C. and stirred for 1 h. After this period, sodium (triacetoxy)borohydride (1.213 g, 5.73 mmol) was added portionwise and the reaction mixture stirred at 25° C. under argon overnight. The reaction mixture was diluted with 10% MeOH in DCM (10 ml) and washed with 0.05M NaOH (10 ml) and brine (10 ml). The organic later was separated, dried (Na₂SO₄) and concentrated in vacuo. The crude product was purified by column chromatography on silica eluting with a 0-10% [(9:1) MeOH:ammonia]/DCM gradient to afford the title compound (435 mg). δH (CDCl₃, 250 MHz) 1.15 (6H, d), 1.83 (2H, t), 2.75 (3H, s), 2.82 (2H, dd), 2.98-3.14 (2H, m), 3.54 (2H, s), 3.85 (3H, s), 7.36 (2H, d), 7.45 (2H, d). MS (ES): MH⁺ 360.

### Description 21

### 5-(4{[(3R,5S)-3,5-Dimethyl-1-piperazinyl]methyl}phenyl)-2-methyl-1,3-thiazole-4-carboxylic acid (D21)

To a solution of D₂₀ (352 mg, 0.98 mmol) in dioxane (10 ml) was added 2M NaOH (10 ml) and the reaction mixture stirred at 25° C. for 90 min. After this period, the reaction mixture was acidified to pH6 with 2M HCl and solvent removed in vacuo to afford title compound as the di-HCl salt (411 mg). δH (DMSO-d₆, 250 MHz) 1.23 (6H, d), 2.67 (3H, s), 2.95-3.05 (2H, m), 3.20-3.80 (6H, m), 7.30-7.55 (4H, m), 8.84 (1H, br s), 9.73 (1H, br s), 12.89 (1H, br s). MS (ES): MH⁺ 346.

### Description 22

### 5-(4-{[(3R,5S)-3,5-Dimethyl-1-piperazinyl]methyl}phenyl)-2-methyl-1,3-thiazole-4-carbaldehyde (D22)

To a stirred solution of D20 (0.345 g, 0.96 mmol) in dry THF (30 ml) at −78° C., under argon was added lithium aluminium hydride (1.15 ml, 1.15 mmol, 1M solution in THF) dropwise. The reaction mixture was stirred at −78° C. for 1 h and then left to warm to 25° C. overnight. The reaction mixture was then quenched with water (1 ml), 2M NaOH (2 ml) and water (1.2 ml), stirred for 30 min and then filtered through Celite. The solid residue obtained was extracted with EtOAc (4×10 ml) and DCM (2×10 ml). The combined organic layers were dried (Na₂SO₄) and concentrated in vacuo to afford the intermediate alcohol which was dissolved in dry DCM (10 ml) then treated with a suspension of [1,1,1-tris(acetyloxy)-1,1-dihydro-1,2-benziodoxol-3-(1H)-one] (449 mg, 1.06 mmol) in dry DCM (5 ml). The reaction mixture was stirred at −78° C. for 45 min. and then kept at 25° C. for 2 h. After this period, a further 0.25 equivalents (83 mg, 0.220 mmol) of 1,1,1-tris(acetyloxy)-1,1-dihydro-1,2-benziodoxol-3-(1H)-one] was added and reaction mixture stirred at 25° C. overnight. After this period, the reaction mixture was quenched with a 1:1 mixture of sodium thiosulphate solution (10% w/v): saturated NaHCO₃ solution (30 ml) and extracted with DCM (3×50 ml). The organic layers were separated, dried (Na₂SO₄) and then concentrated in vacuo. The crude oil was purified by column chromatography on silica eluting with a 0-20% [(9:1) MeOH:ammonia]/DCM gradient to afford the title compound (160 mg). δH (CDCl₃, 250 MHz) 1.09 (6H, d), 1.75 (2H, m), 2.77 (3H, s), 2.81 (2H, m), 2.97-3.04 (2H, m), 3.48 (1H, s), 3.54 (1H, s), 7.37 (2H, d), 7.45 (2H, d), 9.89 (1H, s). MS (ES): MH⁺ 330.

### Description 23

### {[5-(4-[{(3R,5S)-3,5-Dimethyl-1-piperazinyl]methyl}phenyl)-2-methyl-1,3-thiazol-4-yl]methyl}methylamine (D23)

Methylamine (0.25 ml, 0.486 mmol, 2M solution in THF) was added to a stirred solution of D22 (160 mg, 0.486 mmol) in dry MeOH (5 ml) at 25° C. under argon and the reaction mixture stirred for 18 h. After this period, sodium borohydride (27 mg, 0.728 mmol) was added and the reaction stirred for 1 h. On completion, the reaction mixture was partitioned between 2M NaOH (10 ml) and DCM (10 ml). The organic layer was separated and the aqueous re-extracted with DCM (2×10 ml). The combined organic layers were dried (Na₂SO₄) and concentrated in vacuo. The crude oil was purified by chromatography on silica eluting with a 0-20% [(9:1) MeOH:ammonia]/DCM gradient to afford the title compound (92 mg). δH (CDCl₃, 250 MHz) 1.05 (6H, d), 1.66 (2H, t), 2.43 (3H, s), 2.69 (3H, d), 2.78 (2H, d), 2.93-3.00 (2H, m), 3.51 (2H, s), 3.80 (2H, s), 7.30-7.40 (4H, m). MS (ES): MH⁺ 345.

### Description 24

### Methyl 3-(3-fluoro-4-formylphenyl)-2-pyridinecarboxylate (D24)

A suspension of D7 (1.00 g, 3.5 mmol), (3-fluoro-4-formylphenyl)boronic acid (766 mg, 4.6 mmol), tetrakistriphenylphosphine palladium(0) (101 mg, 0.087 mmol), potassium carbonate (630 mg, 4.6 mmol) in toluene (20 ml+4 drops of ionic liquid) were heated in an Emrys™ Optimizer EXP microwave reactor (180° for 10 min). The cooled reaction mixture was diluted with EtOAc, washed with water, saturated NaHCO₃ solution and brine, then dried and concentrated in vacuo. Purification of the resulting crude product on silica, eluting with a 0-100% EtOAc/40-60 petroleum ether gradient, gave the title compound as a white solid (719 mg). δH (CDCl₃, 250 MHz) 3.84 (3H, s), 7.21 (2H, m), 7.57 (1H, dd), 7.78 (1H, dd), 7.93 (1H, t), 8.76 (1H, dd), 10.42 (1H, s).

### Description 25

### Methyl-3-(4-{[(3R,5S)-3,5-dimethyl-1-piperazinyl]methyl}-3-fluorophenyl)-2-pyridine carboxylate (D25)

The title compound was prepared from D24 using a method similar to that described in Description 4.

### Description 26

### 3-(4-{[(3R,5S)-3,5-Dimethyl-1-piperazinyl]methyl}-3-fluorophenyl)-2-pyridinecarboxylic acid (D26)

The title compound was prepared by hydrolysis of D25 using a method similar to that described in Description 5.

### Description 27

### Methyl-3-(4-{[(5S)-5-methyl-1-piperazinyl]methyl}phenyl)-2-pyridine carboxylate (D27)

The title compound was prepared from D3 and (S)-2-methylpiperazine using a method similar to that described in Description 4.

### Description 28

### 4-Bromo-2-methoxybenzonitrile (D28)

4-Bromo-2-fluorobenzonitrile (3.5 g, 17.5 mmol) was dissolved in THF (150 ml) and sodium methoxide (4.73 g, 87.5 mmol) was added. The mixture was heated to 40° C. and stirred for 3 h. After this time the mixture was cooled to 25° C. and Amberlyst 15 resin was added. This mixture was stirred for 3 h, the solid residue was filtered off and the solvent removed in vacuo to give the title compound as a white solid (3.36 g). δH (CDCl₃, 400 MHz) 3.93 (3H, s), 7.16 (1H, s), 7.17 (1H, d), 7.42 (1H, d).

### Description 29

### 4-Bromo-2-methoxybenzene carboxaldehyde (D29)

Ni/Al alloy (5.94 g) was added to a stirred suspension of D28 (3.3 g, 15.5 mmol) in formic acid (55 ml) and water (17 ml). The resulting mixture was heated at 80° C. for 6 h and then allowed to cool. The Ni/Al alloy was filtered off through Celite and the solvent was removed in vacuo. The residue was taken up in water (30 ml) and extracted with EtOAc (30 ml). The organic layer was washed with saturated NaHCO₃ solution, dried (Na₂SO₄) and concentrated in vacuo. Purification of the crude product by column chromatography on silica, eluting with a 0-25% diethyl ether/40-60 petroleum ether gradient, gave the title compound (1.55 g). δH (CDCl₃, 400 MHz) 3.93 (3H, s), 7.17 (2H, m), 7.68 (1H, d), 10.40 (1H, s).

### Description 30

### Methyl 3-(4-formyl-3-methoxyphenyl)-2-pyridinecarboxylate (D30)

D29 (100 mg, 0.47 mmol), potassium acetate (137 mg, 1.40 mmol), PdCl₂(dppf) (19 mg) and 4,4,4′,4′,5,5,5′,5′-octamethyl-2,2′-bi-1,3,2-dioxaborolane (130 mg, 0.51 mmol) were combined in DMF and heated for 2 h at 80° C. The reaction mixture was cooled to 25° C. and D2 (118 mg, 0.46 mmol), a portion of PdCl₂(dppf) (19 mg) and Na₂CO₃ (148 mg, 1.40 mmol) were added. The resultant mixture was stirred at 80° C. for 18 h. The solvent was removed in vacuo and the residue partitioned between DCM and water. The DCM layer was dried (Na₂SO₄) and concentrated to produce a brown oil which was purified by column chromatography on silica. Elution with a 0-50% EtOAc/40-60 petroleum ether gradient gave the title compound as a pale yellow solid (34 mg). δH (CDCl₃, 400 MHz) 3.81 (3H, s), 3.96 (3H, s), 6.96 (1H, s), 7.00 (1H, d), 7.53 (1H, dd), 7.78 (1H, dd), 7.89 (1H, d), 8.74 (1H, dd), 10.50 (1H, s). MS (ES): MH⁺ 272.

### Description 31

### Methyl-3-(4-{[(3R,5S)-3,5-Dimethyl-4-tert-butoxycarbonyl-1-piperazinyl]methyl}-3-methoxyphenyl)-2-pyridine carboxylate (D31)

D30 (34 mg, 0.12 mmol) and (2R,6S)-1-tert-butoxycarbonyl-2,6-dimethylpiperazine (27 mg, 0.12 mmol) (prepared by a method similar to that described in H. Ishida et al, PCT Int. Appl., WO 2003063874, for the preparation of (2R,6R)-dimethyl-1-tert-butoxycarbonylpiperazine) were dissolved in 1,2-dichloroethane (2 ml) and stirred overnight at room temperature. Sodium (triacetoxy)borohydride (40 mg, 0.19 mmol) was added and the mixture stirred overnight at 25° C. The reaction mixture was washed with saturated NaHCO₃ solution and then water. The organic layer was dried and concentrated in vacuo to give the title compound as a yellow oil (59 mg). δH (CDCl₃, 400 MHz) 1.33 (6H, d), 1.47 (9H, s), 2.22 (2H, dd), 2.68 (2H, d), 3.56 (2H, s), 3.79 (3H, s), 3.83 (3H, s), 6.84 (1H, d), 6.94 (1H, dd), 7.48 (1H, dd), 7.57 (1H, d), 7.80 (1H, dd), 8.66 (1H, dd). MS (ES): MH⁺ 470.

### Description 32

### 3-(4{[(3R,5S)-3,5-Dimethyl-4-tert-butoxycarbonyl-1-piperazinyl]methyl}-3-methoxyphenyl)-2-pyridinecarboxylic acid (D32)

D31 (59 mg, 0.12 mmol) in dioxane (2 ml) was treated with lithium hydroxide (11 mg, 0.25 mmol) in water (2 ml) and the resulting mixture stirred at 25° C. for 3 h. The solvent was removed in vacuo and the residue taken up in water. The solution was acidified to pH5 with 2M HCl, then extracted with EtOAc (×3). The organic layers were dried and concentrated to give the title compound as a pale yellow oil (41 mg). δH (CDCl₃, 400 MHz) 1.34 (6H, d), 1.46 (9H, s), 2.35 (2H, m), 2.90 (2H, d), 3.65 (2H, s), 3.82 (3H, s), 4.16 (2H, m), 5.30-6.40 (1H, br), 6.94 (1H, d), 6.98 (1H, dd), 7.44 (1H, d), 7.49 (1H, dd), 7.76 (1H, dd), 8.60 (1H, m). MS (ES): MH⁺ 456.

### Description 33

### (3R,5S)-3,5-Dimethyl-1-({4-[2-({4-[(4-fluorophenyl)amino]piperidin-1-yl}carbonyl)pyridin-3-yl]-3-methoxyphenyl}methyl)-4-tert-butoxycarbonyl-piperazine (D33)

D32 (41 mg, 0.09 mmol), 1-(3-dimethylaminopropyl)-3-ethylcarbodiimide hydrochloride (26 mg, 0.13 mmol) and 1-hydroxybenzotriazole (18 mg, 0.13 mmol) were dissolved in DMF (2 ml) and stirred at 25° C. for 1 h. D11 (17 mg, 0.09 mmol) was added and the mixture stirred at 25° C. for 3 days. The solvent was removed in vacuo and the residue taken up in DCM and washed with saturated NaHCO₃ solution then brine. The DCM layer was dried (Na₂SO₄) and concentrated in vacuo. The crude product was purified by column chromatography on silica eluting with a 0-100% DCM/EtOAc gradient to give the title compound as a pale yellow oil (34 mg). δH (CDCl₃, 400 MHz) 0.70 (1H, m), 1.18 (1H, m), 1.32 (6H, t), 1.47 (9H, s), 1.65 (1H, m), 1.99 (1H, m), 2.21 (2H, m), 2.68 (2H, m), 2.72-2.90 (2H, br m), 3.23 (2H, m), 3.58 (2H, m), 3.83 (3H, s), 4.10 (2H, m), 4.54 (1H, m), 6.44 (2H, dd), 6.54 (2H, t), 7.04 (1H, dd), 7.10 (1H, d), 7.41 (1H, dd), 7.59 (1H, d), 7.81 (1H, dd), 8.62 (1H, m). MS (ES): MH⁺ 632

### Description 34

### (3S)-3-[(4-Fluorophenyl)oxy]pyrrolidine (D34)

The title compound was prepared from (R) 3-hydroxypyrrolidine and 4-fluorophenol using a method similar to that described by S. Komoriya et al., Bioorg. Med. Chem., 2004, 12, 2099-2114.

### Description 35

### (3R)-3-[(4-Fluorophenyl)oxy]pyrrolidine (D35)

The title compound was prepared from (S) 3-hydroxypyrrolidine and 4-fluorophenol using a method similar to that described by S. Komoriya et al., Bioorg Med. Chem., 2004, 12, 2099-2114.

### Description 36

### 3-[(4-Fluorophenyl)amino]pyrrolidine (D36)

The title compound was prepared from pyrrolidin-3-one and 4-fluoroaniline using a method similar to that described by M. Adachi et al., Chem. Pharm. Bull., 1985, 33, 1826-35.

### Description 37

### 2-Pyridinesulfonyl chloride (D37)

Chlorine gas was bubbled through a solution of 2-mercaptopyridine (10 g, 90 mmol) in conc. HCl (70 ml) and water (10 ml) at 0° C. for 2 h. The solution was purged with argon for 15 min, prior to pouring into ice-water. DCM (200 ml) was added and the resulting mixture neutralised with solid NaHCO₃, keeping the temperature at ˜0° C. by addition of ice. The phases were separated and the aqueous layer washed with cold DCM (2×100 ml). The combined organics were dried (Na₂SO₄) and concentrated to give the title compound as a colourless oil (14.8 g) which was either used immediately in the next step, or stored temporarily in the freezer. δH (CDCl₃, 250 MHz) 7.70 (1H, m), 8.07 (1H, m), 8.12 (1H, d), 8.84 (1H, m).

### Description 38

### 8-(2-Pyridinylsulfonyl)-1,4-dioxa-8-azaspiro[4.5]decane (D38)

D37 (10.2 g, 57 mmol) was dissolved in DCM (30 ml) and added via cannula to a solution of 1,4-dioxa-8-azaspiro[4.5]decane (6.9 g, 48.3 mmol) and triethylamine (9.4 ml, 67.5 mmol) in DCM (120 ml). The resultant solution was stirred at room temperature for 18 h. The solution was washed with water (100 ml) and saturated NaHCO₃ solution (100 ml) then dried (Na₂SO₄) and concentrated. Purification by chromatography on silica eluting with 1:1 40-60 petroleum ether/EtOAc gave the title compound as a colourless solid (13.36 g). δH (CDCl₃, 250 MHz) 1.77 (4H, m), 3.46 (4H, m), 3.93 (4H, s), 7.49 (1H, m), 7.88-7.95 (2H, m), 8.72 (1H, m).

### Description 39

### 8-[(3-Iodo-2-pyridinyl)sulfonyl]-1,4-dioxa-8-azaspiro[4.5]decane (D39)

n-Butyllithium (30 ml, 2.5M solution in hexanes, 75.3 mmol) was added dropwise to a solution of diisopropylamine (11.9 ml, 84.7 mmol) in diethyl ether (170 ml) at 0° C. The reaction was stirred for 30 mins and then warmed to room temperature and stirred for a further 30 mins. The resultant solution of lithium diisopropylamine was cooled to −78° C. and a solution of D38 (14.19 g, 50 mmol) in THF (100 ml) was added dropwise over 1.5 h via cannula maintaining the reaction temperature below −70° C. The reaction was stirred for 1 h. Iodine (23.9 g, 94.1 mmol) in THF (60 ml) was added dropwise over 45 mins via cannula, maintaining the reaction temperature below −70° C. and stirring was continued for 1 h. The reaction was quenched by the addition of THF/water (2:1, 75 ml) at −78° C. and then warmed to room temperature. The resultant solution was diluted with EtOAc (300 ml) and a solution of sodium thiosulphate (200 ml). The phases were separated and the organic layer dried (Na₂SO₄) and concentrated. Chromatography on silica eluting with a 0-50% EtOAc/petroleum ether gradient afforded the title compound as a colourless solid (16 g). δH (CDCl₃, 250 MHz) 1.85 (4H, m), 3.69 (4H, m), 4.00 (1H, s), 7.12 (1H, dd), 8.35 (1H, d), 8.56 (1H, d).

### Description 40

### 4-[2-(1,4-Dioxaspiro[4.5]dec-8-ylsulfonyl)-3-pyridinyl]-2-fluorobenzaldehyde (D40)

A degassed mixture of D39 (4.6 g, 11.16 mmol), 3-fluoro-4-formylphenylboronic acid (2.25 g, 13.4 mmol), tetrakis(triphenylphosphine)palladium(0) (258 mg, 0.223 mmol) and sodium carbonate (22.3 ml, 2M solution in water, 44.7 mmol) in 1,2-dimethoxyethane (100 ml) was heated at reflux under argon for 18 h. After cooling the mixture was diluted with EtOAc (200 ml) and washed with water (200 ml). The organic layer was dried (Na₂SO₄) and concentrated. Chromatography on silica eluting with a 30-50% EtOAc/petroleum ether gradient afforded the title compound as a light brown solid (4.48 g). δH (CDCl₃, 250 MHz) 1.81 (4H, m), 3.64 (4H, m), 3.99 (4H, s), 7.37 (2H, m), 7.54 (1H, dd), 7.73 (1H, dd), 7.94 91H, m), 8.67 (1H, d), 10.41 (1H, s).

### Description 41

### (3R,5S)-1-({4-[2-(1,4-Dioxaspiro[4.5]dec-8-ylsulfonyl)-3-pyridinyl]-2-fluorophenyl}methyl)-3,5-dimethylpiperazine (D41)

A mixture of D40 (4.48 g, 11.03 mmol), (2R,6S)-dimethylpiperazine (1.32 g, 11.9 mmol) and sodium (triacetoxy)borohydride (3.5 g, 16.6 mmol) in 1,2-dichloroethane (50 ml) was stirred at room temperature for 6 h. The reaction was quenched by the addition of saturated NaHCO₃ solution (50 ml) and stirred for 15 mins. The phases were separated and the organic layer dried (Na₂SO₄) and concentrated. Chromatography on silica eluting with a 5-10% [2M NH₃ in MeOH]/DCM gradient afforded the title compound as an off white solid (5.5 g). δH (CDCl₃, 250 MHz) 1.05 (6H, d), 1.72 (2H, t), 1.79 (4H, m), 2.82 (2H, dd), 2.96 (2H, m), 3.60 (6H, m), 3.98 (4H, s), 7.17 (1H, dd), 7.26 (1H, dd), 7.45 (1H, t), 7.48 (1H, dd), 7.73 (1H, dd), 8.61 (1H, dd).

### Description 42

### 1-{[3-(4-{[(3R,5S)-3,5-Dimethyl-1-piperazinyl]methyl}-3-fluorophenyl)-2-pyridinyl]sulfonyl}-4-piperidinone (D42)

A solution of D41 (5.5 g, 10.9 mmol) in HCl (6M, 30 ml) and acetone (80 ml) was heated to 55° C. for 3 h. After cooling the reaction was diluted with water (50 ml) and neutralized with potassium carbonate. The resultant solution was extracted with EtOAc (2×, 200 ml) and the organic layers dried (Na₂SO₄) and concentrated to afford the title compound as an off white solid (4.75 g). δH (CDCl₃, 250 MHz) 1.04 (6H, d), 1.75 (2H, t), 2.61 (4H, m), 2.82 (2H, m), 2.92 (2H, m), 3.57 (2H, s), 3.85 (4H, m), 7.23 (2H, m), 7.51 (1H, t), 7.54 (1H, dd), 7.77 (1H, dd), 8.57 (1H, dd).

### Description 43

### N-(3-Fluorophenyl)-1-(1H-imidazol-2-ylcarbonyl)-4-piperidinamine (D43)

Ethyl imidazole-2-carboxylate (701 mg, 5.0 mmol) and D12 (971 mg, 5.0 mmol) were dissolved in toluene (25 ml) and flushed with argon. This solution was cooled to 0° C. and treated with trimethylaluminium (7.5 ml, 2M solution in hexanes, 15 mmol). The mixture was then warmed to 25° C. and stirred for 16 h. The temperature was raised to 50° C. and the mixture stirred for 4 h. The mixture was cooled to 25° C. and stirred for 3 days then treated with Rochelle's salt (20 ml) and stirred for 1 h. The resultant solution was poured into water (30 ml) and extracted with EtOAc (3×40 ml). The combined organics were washed with brine, dried (MgSO₄) and concentrated in vacuo. Purification by column chromatography on silica eluting with a 0-10% MeOH/DCM gradient gave the title compound as a pale yellow solid (775 mg). δH (CDCl₃, 400 MHz) 1.50 (2H, m), 2.21 (2H, d), 3.09 (1H, t), 3.58 (2H, m), 3.68 (1H, m), 4.51 (1H, d), 5.80 (1H, d), 6.25-6.40 (3H, m), 7.09 (1H, q), 7.12 (1H, s), 7.20 (1H, s), 10.72 (1H, br s). MS (ES): MH⁺ 289, (M-H⁺) 287.

### Description 43 (Alternative Procedure)

### N-(3-Fluorophenyl)-1-(1H-imidazol-2-ylcarbonyl)-4-piperidinamine (D43)

Imidazole-2-carboxylic acid (112 mg, 1.0 mmol), 1-(3-dimethylaminopropyl)-3-ethylcarbodiimide hydrochloride (192 mg, 1.0 mmol), 1-hydroxybenzotriazole (135 mg, 1.0 mmol) and triethylamine (303 mg, 3.0 mmol) in DMF (2 ml) was stirred for 30 min. D12 (194 mg, 1.0 mmol) was added and the whole stirred at 25° C. for 16 h then heated to 80° C. for 40 h. The mixture was evaporated to dryness, dissolved in DCM (20 ml) then washed with saturated ammonium chloride solution (20 ml), saturated NaHCO₃ solution (20 ml) and brine (20 ml). The organic phase was then dried (MgSO₄) and concentrated in vacuo. The crude residue was purified by column chromatography on silica eluting with a 0-5% EtOAc/40-60 petroleum ether gradient to give the title compound (20 mg).

### Description 44

### 4-[2-({4-[(3-Fluorophenyl)amino]-1-piperidinyl}carbonyl)-1H-imidazol-1-yl]benzaldehyde (D44)

D43 (775 mg, 2.69 mmol) and (4-formylbenzene)boronic acid (806 mg, 5.38 mmol) were dissolved in 1,2-dichloroethane (13 ml). [Copper(OH)TMEDA]₂Cl₂ (125 mg, 0.27 mmol) was added in a single portion and the resultant mixture heated to 50° C. and stirred for 42 h while bubbling O₂ through the solution. The mixture was cooled to 25° C., diluted with EtOAc and filtered through Keiselghur then evaporated. The crude residue was purified by column chromatography on silica eluting with a 0-5% MeOH/DCM gradient to give the title compound (914 mg). δH (CDCl₃, 400 MHz) 1.45 (2H, m), 2.13 (2H, d), 3.02 (1H, t), 3.35 (1H, t), 3.52 (1H, m), 4.45 (2H, d), 6.29 (1H, d), 6.37 (3H, m), 7.08 (1H, q), 7.19 (1H, s), 7.24 (1H, s), 7.51 (2H, d), 8.00 (2H, d), 10.06 (1H, s). MS (ES): MH⁺ 393.

### Description 45

### Ethyl 1-(4-formylphenyl)-1H-imidazole-2-carboxylate (D45)

Ethyl imidazole-2-carboxylate (140 mg, 1.0 mmol), 4-formylbenzeneboronic acid (300 mg, 2.0 mmol) and catalyst [Copper(OH).TMEDA]₂Cl₂ (46 mg, 0.10 mmol) were dissolved in 1,2-dichloroethane (5 ml) and heated to 60° C. under an O₂ atmosphere for 20 h. The mixture was diluted with DCM, filtered through Celite and evaporated to dryness. The crude residue was purified by column chromatography on silica, eluting with a 0-5% [2M NH₃ in MeOH]/DCM gradient to give the title compound (152 mg). δH (CDCl₃, 250 MHz) 1.32 (3H, t), 4.32 (2H, q), 7.22 (1H, s), 7.34 (1H, s), 7.52 (2H, d), 8.01 (2H, d), 10.11 (1H, s). MS (ES): MH⁺ 245.

### Description 46

### Ethyl 1-(4-{[(3R,5S)-3,5-dimethyl-1-piperazinyl]methyl}phenyl)-1H-imidazole-2-carboxylate (D46)

D45 (152 mg, 0.62 mmol) was dissolved in 1,2-dichloroethane (3 ml), treated with (2R,6S)-dimethylpiperazine (85 mg, 0.75 mmol) in one portion and heated at 60° C. for 3 h. The mixture was cooled to 25° C., sodium (triacetoxy)borohydride (198 mg, 0.93 mmol) was added in one portion and the resultant mixture stirred overnight. The mixture was treated with saturated NaHCO₃ solution (25 ml) and extracted with EtOAc (3×20 ml). The combined organic layers were washed with brine (20 ml), dried (MgSO₄) and evaporated to dryness. The crude residue was purified by column chromatography on silica, eluting with a 0-5% [2M NH₃ in MeOH]/DCM gradient to give the title compound as a colourless oil (102 mg). δH(CDCl₃, 250 MHz) 1.15 (6H, d), 1.31 (3H, t), 1.66 (2H, t), 2.88 (2H, m), 2.97 (2H, m), 3.55 (2H, s), 4.29 (2H, q), 7.17 (1H, s), 7.26 (3H, m), 7.42 (2H, d). MS (ES): MH⁺ 343.

The following intermediates were prepared from the appropriate aniline using the methods outlined in Descriptions 10 and 11.
4-[(3,5-Difluorophenyl)amino]piperidine (D47)
4-[(4-Fluoro-3-methoxyphenyl)amino]piperidine (D48)
4-[(3-Cyanophenyl)amino]piperidine (D49)

### Description 50

### 1-(tert-Butoxycarbonyl)-4-(3-fluorophenoxy)piperidine (D50)

To a solution of 1-(tert-butoxycarbonyl)-4-hydroxypiperidine (24 g, 112 mmol), 3-fluorophenol (5.6 g, 59 mmol) and triphenylphosphine (31.4 g, 118 mmol) in tetrahydrofuran (100 ml) was added di-isopropylazodicarboxylate (23.3 ml, 118 mmol) at 0° C. The reaction was stirred at room temperature for 24 h and then the solvent concentrated in vacuo. The resultant yellow oil was diluted with dichloromethane (5 ml) and hexane (200 ml), stirred for 30 min and the resultant white precipitate filtered off. The filtrate was concentrated in vacuo and purified by column chromatography on silica (dichloromethane) to give the title compound as a yellow oil (14.5 g). δH (CDCl₃, 250 MHz) 1.47 (9H, s), 1.76 (2H, m), 1.92 (2H, m), 3.35 (2H, ddd), 3.69 (2H, ddd), 4.44 (1H, m), 6.65 (3H, m), 7.20 (1H, m).

### Description 51

### 4-(3-Fluorophenoxy)piperidine (D51)

A solution of 1-(tert-butoxycarbonyl)-4-(3-fluorophenoxy)piperidine (D50) (14.5 g, 55 mmol) in dichloromethane (200 ml) at 0° C. was treated with trifluoroacetic acid (17 ml). The reaction was warmed to room temperature and stirred for 16 h. The solvent was then concentrated in vacuo and the residue partitioned between dichloromethane and 2M NaOH solution. The organics were extracted with 2M HCl (×2) which was then basified with 2M NaOH and re-extracted with dichloromethane (×3). The combined organics were dried (MgSO₄) and concentrated in vacuo to give the title compound as a yellow oil (8.0 g). δH (CDCl₃, 250 MHz) 1.66 (2H, m), 2.01 (2H, m), 2.73 (2H, m), 3.14 (2H, m), 4.34 (1H, m), 6.68 (3H, m), 7.19 (1H, m).

### Description 52

### 1-(Diphenylmethyl)-3-azetidinone (D52)

The title compound was prepared from 1-(Diphenylmethyl)-3-azetidinol using a method similar to that described in A. R. Katritzky et al, J. Heterocyclic Chem., 1994, 31(2), 271-275.

### Description 53

### 1-Chloroethyl 3-oxo-1-azetidinecarboxylate (D53)

The title compound was prepared from D52 using a method similar to that described in F-W. Sum et al, Bioorg. Med. Chem. Lett., 2003, 13, 2191-2194.

### Description 54

### N-(4-Fluorophenyl)-3-azetidinamine (D54)

A mixture of D53 (4.2 mmol) and 4-fluoroaniline (467 mg, 4.2 mmol) in 1,2-dichloroethane (50 ml) under an argon atmosphere was stirred at 50° C. for 4 h. The mixture was then cooled to 0° C. and sodium (triacetoxy)borohydride (1.78 g, 8.4 mmol) was added. The reaction was then stirred at room temp. for 3 days then diluted with DCM. The organics were washed with 0.05M NaOH and brine, then dried and concentrated. The crude intermediate was then taken up in MeOH (50 ml) and heated at reflux for 1 h. On cooling, the solvent was removed in vacuo and the residue partitioned between DCM at sat. NaHCO₃ solution. The organic phase was dried and concentrated in vacuo to give the crude product which was purified by column chromatography on silica. Elution with a 0-10% (2M ammonia in MeOH)/DCM gradient gave the title compound as a brown oil (39 mg). δH (CDCl₃, 250 MHz) 3.01 (1H, br s), 3.47 (2H, br s), 3.93 (2H, br s), 4.03 (1H, br s), 4.27 (1H, br s), 6.47 (2H, dd), 6.88 (2H, t). MS (ES): MH⁺ 167.

### Description 55

### 2-({4-[(4-Fluorophenyl)amino]-1-piperidinyl}carbonyl)-3-pyridinol (D55)

To a mixture of 3-hydroxy-2-pyridinecarboxylic acid (4.36 g, 31.4 mmol) and N-methylmorpholine (6.9 ml, 62.8 mmol) in dry THF (150 ml), cooled to 0° C., was added a solution of isobutyl chloroformate (8.16 ml, 62.8 mmol) in dry THF (70 ml) over a period of 5 mins. After stirring at 0° C. for 1 h, a solution of D11 (5.79 g, 29.8 mmol) in dry THF (40 ml) was added over 10 mins. After stirring for a further 1 h the mixture was treated with 2M NaOH solution (100 ml), warmed to room temp then neutralised with c.HCl. The THF was removed in vacuo and the resulting solid precipitate was collected by filtration washed with water then dried to give the title compound (9.25 g). δH (DMSO, 400 MHz) 1.30 (2H, m), 1.80-1.99 (2H, m), 3.06 (2H, m), 3.34-3.49 (2H, m), 4.37 (1H, m), 5.47 (1H, d), 6.58 (2H, m), 6.90 (2H, t), 7.26 (2H, m), 8.03 (1H, dd), 10.30 (1H, s). MS (ES): MH⁺ 316.

### Description 56

### 2-({4-[(4-Fluorophenyl)methyl]-1-piperidinyl}carbonyl)-3-iodopyridine (D56)

The title compound was prepared from D1 and 4-[(4-fluorophenyl)methyl]piperidine using a method similar to that described in Description 15.

### Description 57

### 4-[2-({4-[(4-fluorophenyl)methyl]-1-piperidinyl}carbonyl)-3-pyridinyl]benzaldehyde (D57)

The title compound was prepared from D56 and 4-formylbenzene boronic acid using a method similar to that described in Description 3.

### EXAMPLE 1

### (3R,5S)-3,5-Dimethyl-1-({4-[2-({4-[(4-Fluorophenyl)methyl]piperidin-1-yl}carbonyl)pyridin-3-yl]phenyl}methyl)piperazine (E1)

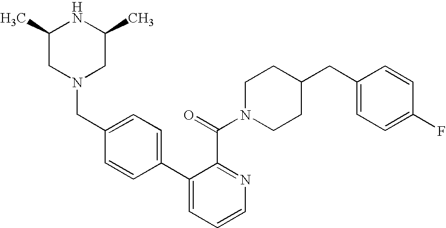

A mixture of D57 (63 mg, 0.157 mmol), (2R,6S)-2,6-dimethylpiperazine (36 mg, 0.314 mmol) and sodium (triacetoxy)borohydride (66 mg, 0.314 mmol) in DCM (5 ml) was stirred under at argon atmosphere at room temp for 1 day. The mixture was diluted with DCM (10 ml) and washed with 0.05M NaOH solution. The aqueous phase was extracted with DCM (20 ml) and the combined organics were concentrated in vacuo to give the crude product which was purified by column chromatography on silica. Elution with 10% (9:1 MeOH/ammonia) in DCM gave the title compound (16 mg). δH (CDCl₃, 250 MHz) 1.07 (6H, d), 1.24 (2H, d), 1.51-1.63 (2H, m), 1.76 (2H, t), 2.22-2.35 (4H, m), 2.43-2.64 (4H, m), 2.80 (2H, m), 2.98-3.06 (2H, m), 3.18 (1H, m), 4.63 (1H, m), 6.88-7.01 (4H, m), 7.36-7.55 (5H, m), 7.76 (1H, dd), 8.62 (1H, dd). MS (ES): C₃₁H₃₇FN₄O requires 500. found 501 (MH⁺).

### EXAMPLE 2

### (3R,5S)-3,5-Dimethyl-1-({4-[2-({4-[(4-fluorophenyl)amino]piperidin-1-yl}carbonyl)pyridin-3-yl]phenyl}methyl)piperazine (E2)

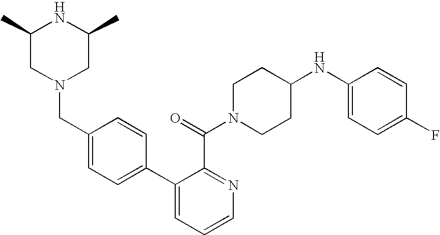

A mixture of D5 (35 mg, 0.107 mmol), 1-(3-dimethylaminopropyl)-3-ethylcarbodiimide hydrochloride (23 mg, 0.118 mmol), 1-hydroxybenzotriazole (16 mg, 0.118 mmol), triethylamine (60 μl, 0.438 mmol) and D11 (19 mg, 0.107 mmol) in dry DMF (3 ml) was heated at 80° C. for 18 h. After this period, the mixture was concentrated in vacuo and the residue partitioned between saturated NaHCO₃ solution and 10% MeOH in DCM (10 ml). The organic layer was separated and the aqueous layer was re-extracted with 10% MeOH in DCM. The combined organic layers were dried (Na₂SO₄), then concentrated in vacuo and the resulting residue was purified by column chromatography on silica eluting with a 0-10% [(9:1) MeOH:ammonia]/DCM gradient to afford the title compound as a pale yellow oil (16 mg). MS (ES): MH⁺ 502.

### EXAMPLE 2

### Alternative Procedure

### (3R,5S)-3,5-Dimethyl-1-({4-[2-({4-[(4-fluorophenyl)amino]piperidin-1-yl}carbonyl)pyridin-3-yl]phenyl}methyl)piperazine (E2)

D15 (0.92 g, 1.53 mmol) was dissolved in trifluoroacetic acid (6 ml) and DCM (24 ml) and stirred at 25° C. for 1 h then evaporated to dryness. The residue (as the TFA salt) was partitioned between DCM and saturated NaHCO₃ solution. The organic phase was separated, dried and concentrated in vacuo to give the title compound as a solid (0.771 g). MS (ES): C₃₀H₃₆FN₅O requires 501. found 502 (MH⁺).

This whole was dissolved in EtOAc and treatment with 1.2M ethereal HCl (1.28 ml) afforded (3R,5S)-3,5-dimethyl-1-({4-[2-({4-[(4-fluorophenyl)amino]piperidin-1-yl}-carbonyl)pyridin-3-yl]phenyl}methyl)piperazine hydrochloride as a white solid. δH (MeOH-d₄, 250 MHz) 1.35 (6H, m), 1.40-1.65 (2H, m), 1.83 (1H, d), 2.04 (1H, d), 2.58 (2H, br), 2.79 (1H, m), 2.99 (1H, m), 3.20-3.80 (6H, overlapping signals), 4.04 (2H, br), 4.63 (1H, d), 7.25 (2H, t), 7.36 (2H, br), 7.50 (2H, d), 7.61 (3H, m), 7.97 (1H, dd), 8.62 (1H, m). MS (ES): C₃₀H₃₆FN₅O requires 501. found 502 (MH⁺)

### EXAMPLE 2

### Alternative Procedure

### (3R,5S)-3,5-Dimethyl-1-({4-[2-({4-[(4-fluorophenyl)amino]piperidin-1-yl}carbonyl)pyridin-3-yl]phenyl}methyl)piperazine (E2)

A suspension of D55 (1 g, 3.17 mmol) in dry DCM (25 ml) was treated with triethylamine (0.485 ml, 3.49 mmol) and 1,1,1-trifluoro-N-phenyl-N-[(trifluoromethyl)sulfonyl]methanesulfonamide (1.25 g, 3.49 mmol) and the resulting solution stirred at room temp. overnight. The reaction mixture was then concentrated in vacuo to give 2-({4-[(4-fluorophenyl)amino]-1-piperidinyl}carbonyl)-3-pyridinyl trifluoromethanesulfonate as a crude pale grey gum/foam, MS (ES): MH⁺ 448. This whole was combined with 4-formylbenzene boronic acid (475 mg, 3.81 mmol), tetrakis(triphenylphosphine)palladium (0) (366 mg, 0.317 mmol) and sodium carbonate (672 mg, 6.34 mmol) in a mixture of water (6 ml) and 1,2-DME (17 ml), under an argon atmosphere. The mixture was heated at 60° C. for 4 h then cooled to room temp. overnight. The mixture was concentrated in vacuo to remove the 1,2-DME and the residue partitioned between EtOAc and water. The organic phase was washed with a 10% aqueous solution of L-cysteine, brine and then dried and concentrated in vacuo to give 4-[2-({4-[(4-fluorophenyl)amino]-1-piperidinyl}carbonyl)-3-pyridinyl]benzaldehyde as a crude pale yellow gum/foam (2.23 g), MS (ES): MH⁺ 404. This whole was taken up in 1,2-dichloroethane (25 ml) and (2R,6S)-2,6-dimethylpiperazine (435 mg, 3.81 mmol) was added. The mixture was heated at 50° C. for 3 h, cooled to room temp., sodium (triacetoxy)borohydride (1.34 g, 6.34 mmol) was added and the reaction stirred at room temp. for 3 days. The reaction mixture was diluted with 2N HCl (20 ml), stirred vigorously for 0.5 h then basified to pH11-12 with 50% aq. NaOH solution. The organic phase was separated and the aqueous phase re-extracted with DCM. The combined organics were washed with brine, dried and concentrated in vacuo to give the crude product which was purified by column chromatography on silica. Elution with an ammonia/MeOH/DCM gradient afforded the title compound as a foam (0.83 g). δH (CDCl₃, 250 MHz) 0.72 (1H, m), 1.04 (6H, d), 1.15 (1H, m), 1.61-1.72 (4H, m), 1.97 (1H, br d), 2.74-3.00 (6H, m), 3.20-3.25 (3H, m), 3.56 (2H, s), 4.51 (1H, m), 6.45 (2H, m), 6.84 (2H, t), 7.40 (3H, m), 7.46 (2H, d), 7.77 (1H, dd), 8.62 (1H, dd). MS (ES): MH⁺ 502.

### EXAMPLE 3

### N-{[3-(4{[(3R,5S)-3,5-Dimethyl-1-piperazinyl]methyl}phenyl)-2-pyridinyl]methyl}-3-(4-fluorophenyl)-N-methylpropanamide (E3)

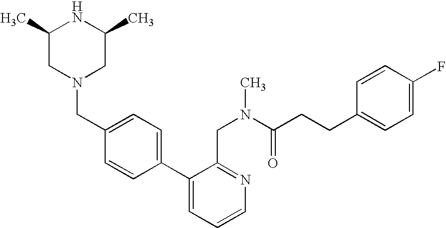

A suspension of 3-(4-fluorophenyl)propanoic acid (17 mg, 0.111 mmol), N-cyclohexylcarbodiimide N′-methyl polystyrene (Novabiochem, 57 mg, 0.102 mmol, loading 1.7 mmol/g) and 1-hydroxybenzotriazole (14 mg, 0.102 mmol) in dry DCM (2 ml) was stirred at 25° C. for 1 h. D18 (30 mg, 0.093 mmol) was added in dry DCM (2 ml). The reaction mixture was stirred overnight before polymer supported trisamine resin (Argonaut, 22 mg, 0.093 mmol, loading 4.17 mmol/g) was added and the reaction mixture stirred for a further 1 h. After this period, the mixture was filtered and the solvent removed in vacuo. The residue was purified by column chromatography on silica eluting with a 0-10% MeOH/EtOAc gradient followed by a 0-10% [(9:1)MeOH:ammonia]/EtOAc gradient to afford the title compound (18 mg, 41%). δH (CDCl₃, 250 MHz) 1.04 (6H, d), 1.65 (2H, t), 2.40-2.60 (2H, m), 2.75-2.95 (9H, m), 3.53 (1H, s), 3.54 (1H, s), 4.49 (1H, s), 4.73 (1H, s), 6.90-7.00 (2H, m), 7.00-7.56 (8H, m), 8.53-8.59 (1H, m). MS (ES): C₂₉H₃₅FN₄O requires 474. found 475 (MH⁺)

### EXAMPLE 4

### (3R,5S)-3,5-Dimethyl-1-({4-[4-({4-[(4-fluorophenyl)methyl]piperidin-1-yl}carbonyl)-2-methyl-1,3-thiazol-5-yl]phenyl}methyl)piperazine (E4)

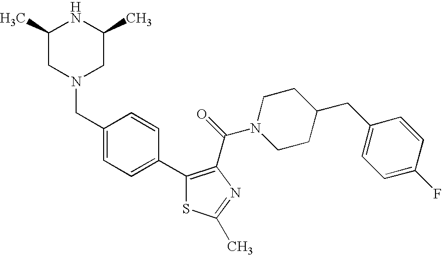

To a suspension of D21 (0.03 g, 0.09 mmol), 1-(3-dimethylaminopropyl)-3-ethylcarbodiimide hydrochloride (0.023 g, 0.12 mmol) and 1-hydroxybenzotriazole (0.014 g, 0.11 mmol) in dry DMF (5 ml) was added by 4-[(4-fluorophenyl)methyl]piperidine (0.027 g, 0.14 mmol) in dry DMF (2 ml). The resultant suspension was heated at 50° C. under argon for 18 h. The reaction mixture was then concentrated in vacuo and the crude product purified by column chromatography on silica eluting with a 0-20% [(9:1) MeOH:ammonia)/DCM gradient to afford the title compound (11.7 mg, 26%). δH (MeOH-d₄, 400 MHz) 0.84 (1H, m), 1.05-1.37 (2H, m), 1.45 (6H, m), 1.67-1.85 (2H, m), 2.47 (2H, m), 2.75 (3H, s), 2.70-3.00 (2H, m), 3.20-3.60 (4H, m), 3.70 (2H, m), 3.90 (2H, m), 4.59 (2H, m), 6.97 (2H, t), 7.14 (2H, dd), 7.60 (2H, d), 7.76 (2H, d). MS (ES): C₃₀H₃₇N₄OSF requires 520. found 521 (MH⁺)

The following example was also prepared from D21, together with the amine D11, using a method similar to that described in Example 4.

Compounds possess the general structure:

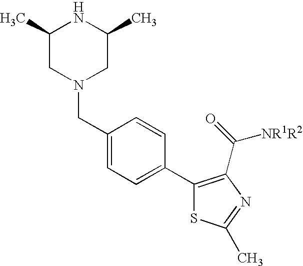

Where NR¹R² is exemplified in the table below

| Example | Compound | NR¹R² | MH⁺ |
|---|---|---|---|
| 5 | (3R, 5S)-3,5-dimethyl-1-({4-[4-({(4-[(4-fluorophenyl)amino]piperidin-1-yl)carbonyl)-2-methyl-1,3-thiazol-5-yl]phenyl}methyl)piperazine (E5) | 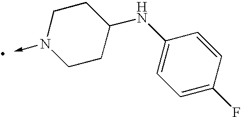 | 522 |

### EXAMPLE 6

### N-{[5-(4-{[(3R,5S)-3,5-Dimethyl-1-piperazinyl]methyl}phenyl)-2-methyl-1,3-thiazol-4-yl]methyl}-3-(4-fluorophenyl)-N-methylpropanamide dihydrochloride (E6)

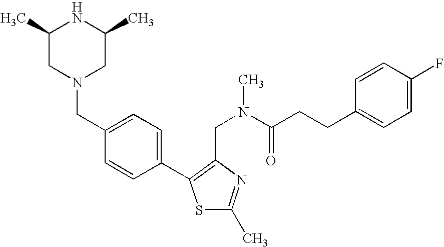

To a suspension of 3-(4-fluorophenyl)propanoic acid (22 mg, 0.131 mmol), 1-(3-dimethylaminopropyl)-3-ethylcarbodiimide hydrochloride (0.023 g, 0.12 mmol) and 1-hydroxybenzotriazole (0.014 g, 0.11 mmol) in dry DCM (5 ml) was added D23 (30 mg, 0.087 mmol) and the reaction mixture stirred under argon at 25° C. for 48 h. The reaction mixture was then diluted with DCM (10 ml) and water (10 ml). The organic layer was separated and the aqueous layer re-extracted with DCM (2×10 ml). The combined organic layers were washed with 2M NaOH and brine, dried (Na₂SO₄) and concentrated in vacuo. Purification by column chromatography on silica eluting with a 0-10% MeOH/EtOAc gradient followed by a 0-10% [(9:1) MeOH:ammonia]/EtOAc gradient gave the free-base, which was treated with 2.2 equivalents of 1M HCl in diethyl ether to give title compound (22 mg, 51%). δH (DMSO-d₆, 400 MHz) 1.29 (6H, d), 2.50-3.10 (12H, m), 3.45-3.55 (2H, m), 4.25-4.35 (2H, m), 4.59 (2H, d), 7.05-7.11 (2H, m), 7.14-7.18 (2H, m), 7.49 (1H, d), 7.60 (1H, d), 7.71 (2H, m), 9.91 (2H, bs). MS (ES): C₂₈H₃₅FN₄OS requires 494. found 495 (MH⁺)

The following example was also prepared from D23 and the appropriate acid following the method similar to that described in Example 6.

Compounds possess the general structure:

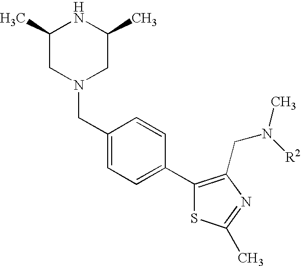

Where R is exemplified in the table below

| Example | Compound | R² | MH⁺ |
|---|---|---|---|
| 7 | N-{[5-(4-{[(3R,5S)-3,5-dimethyl-1-piperazinyl]methyl}phenyl)-2-methyl-1,3-thiazol-4-yl] methyl}-2-[(4-fluorophenyl)oxy]-N-methylacetamide (E7) | 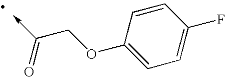 | 497 |

### EXAMPLE 8

### N-(4-Fluorophenyl)-1-{[3-(4-{[(3S)-3-methyl-1-piperazinyl]methyl}phenyl)-2-pyridinyl]carbonyl}-4-piperidinamine (E8)

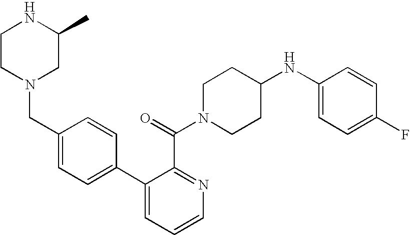

To a solution of D27 (100 mg, 0.31 mmol) in toluene (2.5 ml), under an atmosphere of argon, was added D11 (59.7 mg, 0.31 mmol) followed by dropwise addition of trimethylaluminium (0.46 ml, 2M in solution in toluene, 0.92 mmol). The mixture was stirred at 25° C. for 48 h, diluted with water (5 ml), stirred for 30 min and a further portion of water (20 ml) added. Extraction with EtOAc and concentration in vacuo gave a brown oil (101 mg) which was purified by mass-directed autopreparation to give the title compound (22 mg). MS (ES): MH⁺ 488.

Treatment of this whole with 1.2M ethereal HCl (37 ul) in EtOAc afforded the hydrochloride salt of the title compound (20 mg). δH (DMSO-d₆, 250 MHz) 0.90 (1H, m), 1.09 (1H, m), 1.15 (3H, d), 1.57 (1H, m), 1.85 (1H, m), 2.08 (1H, t), 2.28 (1H, m), 2.70-3.00 (4H, m), 3.10-3.40 (4H, m), 3.60 (2H, m), 4.21 (1H, m), 5.32 (1H, m), 6.53 (2H, m), 6.87 (2H, t), 7.42 (4H, m), 7.54 (1H, dd), 7.91 (1H, d), 8.51 (1H, br), 8.58 (1H, d). MS (ES): C₂₉H₃₄FN₅O requires 487. found 488 (MH⁺).

The following examples E9-E19 were prepared following methods similar to those described above for Examples E1-E8.

| Example | Structure | Compound Name | MH+ |
|---|---|---|---|
| 9 | 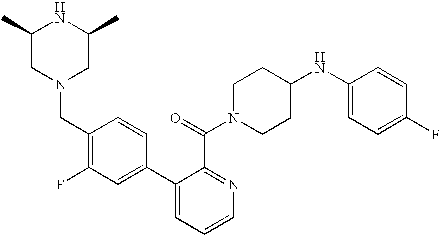 | 1-{[3-(4-{[(3R,5S)-3,5-dimethyl-1-piperazinyl]methyl}-3-fluorophenyl)-2-pyridinyl]carbonyl}-N-(4-fluorophenyl)-4-piperidinamine | 520 |
| 10 | 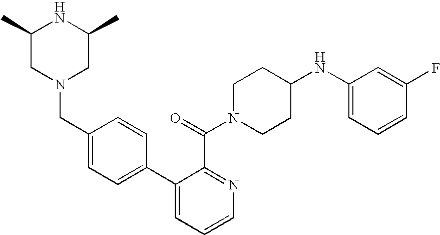 | 1-{[3-(4-{[(3R,5S)-3,5-dimethyl-1-piperazinyl]methyl}phenyl)-2-pyridinyl]carbonyl}-N-(3-fluorophenyl)-4-piperidinamine | 502 |
| 11 | 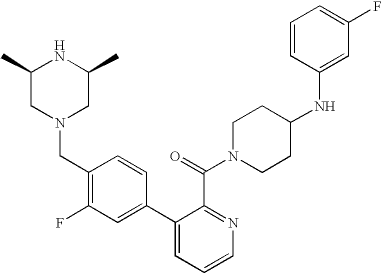 | 1-{[3-(4-{[(3R,5S)-3,5-dimethyl-1-piperazinyl]methyl}-3-fluorophenyl)-2-pyridinyl]carbonyl}-N-(3-fluorophenyl)-4-piperidinamine | 520 |
| 12 | 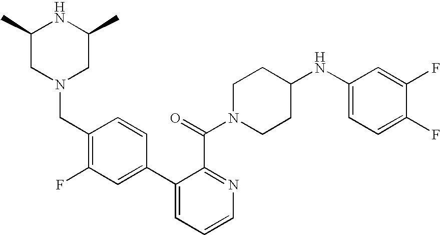 | N-(3,4-difluorophenyl)-1-{[3-(4-{[(3R,5S)-3,5-dimethyl-1-piperazinyl]methyl}-3-fluorophenyl)-2-pyridinyl]carbonyl}-4-piperidinamine | 538 |
| 13 | 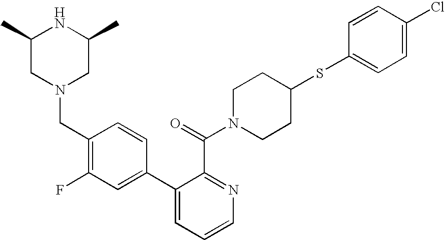 | (3R,5S)-1-({4-[2-({4-[(4-chlorophenyl)thio]-1-piperidinyl}carbonyl)-3-pyridinyl]-2-fluorophenyl}methyl)-3,5-dimethylpiperazine | 554 |
| 14 | 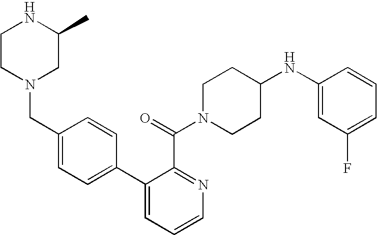 | N-(3-fluorophenyl)-1-{[3-(4-{[(3S)-3-methyl-1-piperazinyl]methyl}phenyl)-2-pyridinyl]carbonyl}-4-piperidinamine | 488 |
| 15 | 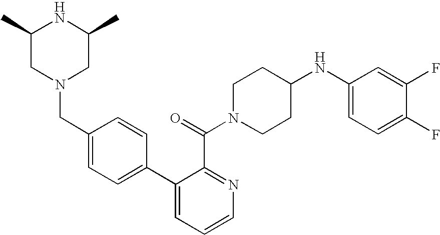 | N-(3,4-difluorophenyl)-1-{[3-(4-{[(3R,5S)-3,5-dimethyl-1-piperazinyl]methyl}phenyl)-2-pyridinyl]carbonyl}-4-piperidinamine | 520 |
| 16 | 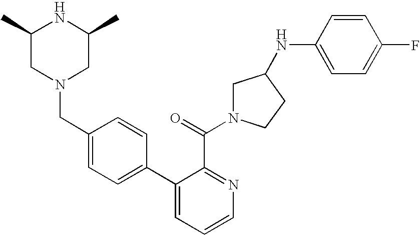 | 1-{[3-(4-{[(3R,5S)-3,5-dimethyl-1-piperazinyl]methyl}phenyl)-2-pyridinyl]carbonyl}-N-(4-fluorophenyl)-3-pyrrolidinamine | 488 |
| 17 | 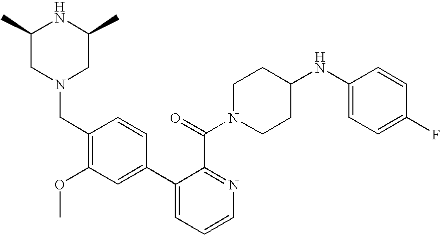 | 1-({3-[4-{[(3R,5S)-3,5-dimethyl-1-piperazinyl]methyl}-3-methyloxy)phenyl]-2-pyridinyl}carbonyl)-N-(4-fluorophenyl)-4-piperidinamine | 532 |
| 18 | 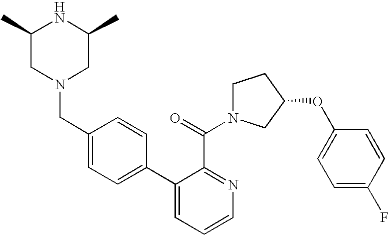 | (3R,5S)-1-({4[2-({(3S)-3-[(4-fluorophenyl)oxy]-1-pyrrolidinyl}carbonyl)-3-pyridinyl]phenyl}methyl)-3,5-dimethylpiperazine | 489 |
| 19 | 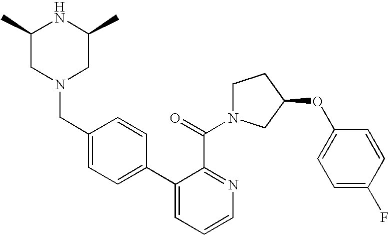 | (3R,5S)-1-({4[2-({(3R)-3-[(4-fluorophenyl)oxy]-1-pyrrolidinyl}carbonyl)-3-pyridinyl]phenyl}methyl)-3,5-dimethylpiperazine | 489 |

### EXAMPLE 20

### 1-{[3-(4-{[(3R,5S)-3,5-Dimethyl-1-piperazinyl]methyl}-3-fluorophenyl)-2-pyridinyl]sulfonyl}-N-(2-fluorophenyl)-4-piperidinamine (E20)

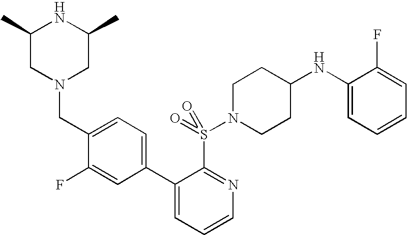

A mixture of D42 (75 mg, 0.163 mmol), 2-fluoroaniline (16 μl, 0.171 mmol) sodium (triacetoxy)borohydride (52 mg, 0.245 mmol) and acetic acid (9 μl, 0.163 mmol) in 1,2-dichloroethane (2 ml) was stirred at room temperature for 18 h. The reaction was quenched by the addition of saturated NaHCO₃ solution (1 ml) and stirred for 15 mins. The organic layer was decanted off onto a phase separation cartridge (Whatman) and the aqueous layer washed with DCM. The combined organic layers were concentrated in vacuo to give the crude product was purified by column chromatography on silica. Elution with a 0-10% MeOH/DCM gradient afforded the title compound as a colourless glass (19 mg). δH (CDCl₃, 250 MHz) 1.06 (6H, d), 1.61 (2H, m), 1.75 (2H, t), 2.12 (2H, m), 2.82 (2H, d), 2.98 (2H, m), 3.30 (2H, m), 3.62 (2H, s), 3.89 (3H, m), 6.64 (2H, m), 7.10 (2H, m), 7.25 (2H, m), 7.49 (1H, t), 7.51 (1H, dd), 7.75 (1H, dd), 8.61 (1H, dd).

### EXAMPLE 21

### 1-{[1-(4-{[(3R,5S)-3,5-Dimethyl-1-piperazinyl]methyl}phenyl)-1H-imidazol-2-yl]carbonyl}-N-(3-fluorophenyl)-4-piperidinamine (E21)

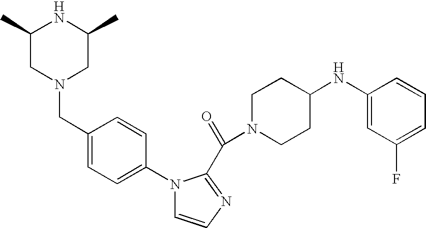

Aldehyde D44 (914 mg, 2.33 mmol) and (2R,6S)-dimethylpiperazine (399 mg, 3.49 mmol) were dissolved in 1,2-dichloroethane (12 ml). Acetic acid (140 mg, 2.33 mmol) was added and the mixture heated at 50° C. for 2 h. After cooling to 25° C., sodium (triacetoxy)borohydride (1.48 g, 6.99 mmol) was added and the reaction was stirred for 16 h. The mixture was treated with saturated NaHCO₃ solution (20 ml), stirred for 1 h then poured into water (50 ml) and extracted with DCM (3×50 ml). The combined organic extracts were washed with brine, dried (MgSO₄) and evaporated to dryness. The residue was purified by column chromatography on silica eluting with a 0-5% (2M ammonia in MeOH)/DCM gradient to give the title compound as an oil (595 mg). δH (CDCl₃, 400 MHz) 1.05 (6H, d), 1.36 (2H, m), 1.65 (2H, t), 2.08 (2H, m), 2.77 (2H, d), 2.95 (3H, m), 3.25 (1H, t), 3.49 (1H, m), 3.53 (2H, s), 3.65 (1H, d), 4.23 (1H, br d), 4.47 (1H, br d), 6.26 (1H, m), 6.36 (3H, m), 7.09 (1H, q), 7.14 (1H, s), 7.18 (1H, s), 7.27 (2H, d), 7.42 (2H, d). MS (ES): C₂₈H₃₅FN₆O requires 490. found 491 (MH⁺).

### EXAMPLE 22

### 1 {[1-(4-{[(3R,5S)-3,5-Dimethyl-1-piperazinyl]methyl}phenyl)-1H-imidazol-2-yl]carbonyl}-N-(4-fluorophenyl)-4-piperidinamine (E22)

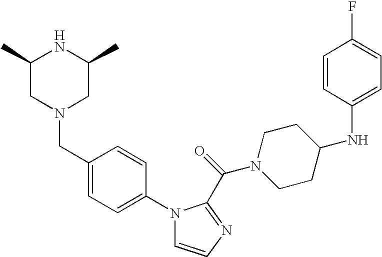

Ester D46 (30 mg, 0.09 mmol) was dissolved in dry toluene (0.5 ml) under an atmosphere of argon. D11 (17 mg, 0.09 mmol) was added in one portion followed by the dropwise addition of the trimethylaluminium (131 ul, 2M solution in hexanes, 0.26 mmol) and the mixture stirred at 25° C. overnight. The mixture was treated with water (3 ml), stirred for 10 min then partitioned between water (20 ml) and EtOAc (20 ml). The aqueous layer was extracted with EtOAc (2×20 ml) and the combined organics washed with brine (20 ml), dried (MgSO₄) and evaporated to dryness. The residue was purified by column chromatography on silica eluting with a 0-10% (2M ammonia in MeOH)/DCM gradient to give the title compound as a colourless oil (13 mg). δH (CDCl₃, 400 MHz) 1.03 (6H, d), 1.32 (2H, m), 1.65 (2H, t), 2.08 (2H, m), 2.78 (2H, m), 2.95 (3H, m), 3.25 (1H, t), 3.42 (2H, m), 3.52 (2H, s), 4.24 (1H, br d), 4.49 (1H, br d), 6.53 (2H, dd), 6.88 (2H, t), 7.15 (1H, s), 7.17 (1H, s), 7.29 (2H, d), 7.40 (2H, d). MS (ES): C₂₈H₃₅FN₆O requires 490. found 491 (MH⁺).

The following examples 23-26 were prepared following methods similar to those described in Examples E21 and E22.

| Example | Structure | Compound Name | MH+ |
|---|---|---|---|
| 23 | 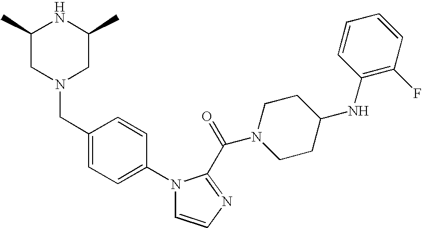 | 1-{[1-(4-{[(3R,5S)-3,5-dimethyl-1-piperazinyl]methyl}phenyl)-1H-imidazol-2-yl]carbonyl}-N-(2-fluorophenyl)-4-piperidinamine | 491 |
| 24 | 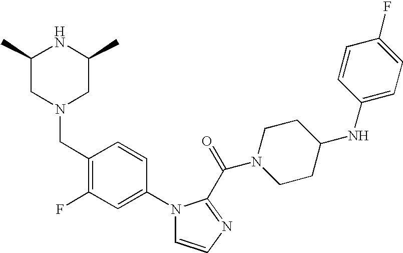 | 1-{[1-(4-{[(3R,5S)-3,5-dimethyl-1-piperazinyl]methyl}-3-fluorophenyl)-1H-imidazol-2-yl]carbonyl}-N-(4-fluorophenyl)-4-piperidinamine | 509 |
| 25 | 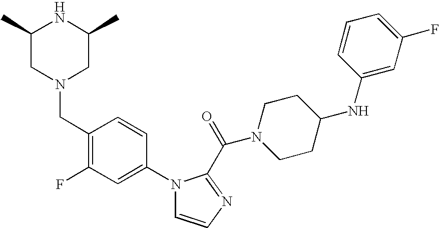 | 1-{[1-(4-{[(3R,5S)-3,5-dimethyl-1-piperazinyl]methyl}-3-fluorophenyl)-1H-imidazol-2-yl]carbonyl}-N-(3-fluorophenyl)-4-piperidinamine | 509 |
| 26 | 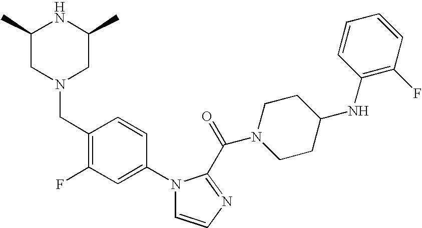 | 1-{[1-(4-{[(3R,5S)-3,5-dimethyl-1-piperazinyl]methyl}-3-fluorophenyl)-1H-imidazol-2-yl]carbonyl}-N-(2-fluorophenyl)-4-piperidinamine | 509 |

The following examples E27-E36 were prepared following methods similar to those described above for Examples E1-E8.

| Example | Structure | Compound Name | MH+ |
|---|---|---|---|
| 27 | 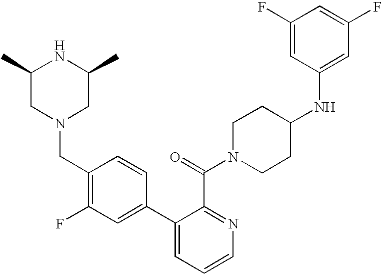 | N-(3,5-difluorophenyl)-1-{[3-(4-{[(3R,5S)-3,5-dimethyl-1-piperazinyl]methyl}-3-fluorophenyl)-2-pyridinyl]carbonyl}-4-piperidinamine | 538 |
| 28 | 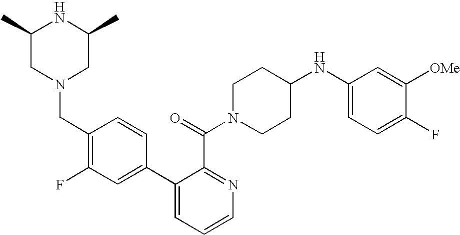 | 1-{[3-(4-{[(3R,5S)-3,5-dimethyl-1-piperazinyl]methyl}-3-fluorophenyl)-2-pyridinyl]carbonyl}-N-[4-fluoro-3-(methyloxy)phenyl]-4-piperidinamine | 550 |
| 29 | 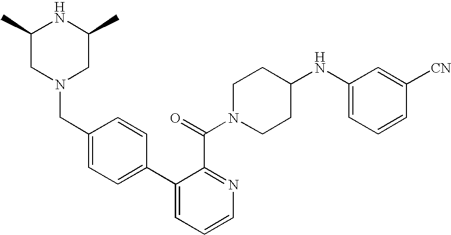 | 3-[(1-{[3-(4-{[(3R,5S)-3,5-dimethyl-1-piperazinyl]methyl}phenyl)-2-pyridinyl]carbonyl}-4-piperidinyl)amino]benzonitrile | 509 |
| 30 | 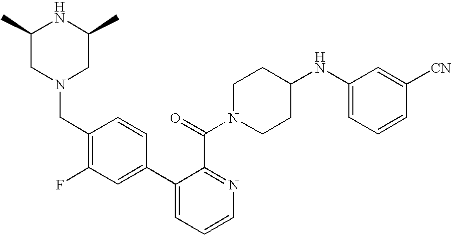 | 3-[(1-{[3-(4-{[(3R,5S)-3,5-dimethyl-1-piperazinyl]methyl}-3-fluorophenyl)-2-pyridinyl]car-bonyl}-4-piperidinyl)amino]benzonitrile | 527 |
| 31 | 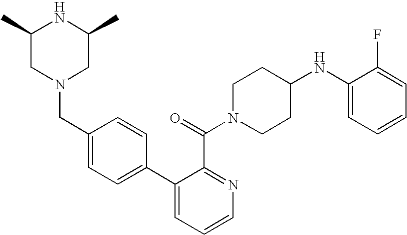 | 1-{[3-(4-{[(3R,5S)-3,5-dimethyl-1-piperazinyl]methyl}phenyl)-2-pyridinyl]carbonyl}-N-(2-fluorophenyl)-4-piperidinamine | 502 |
| 32 | 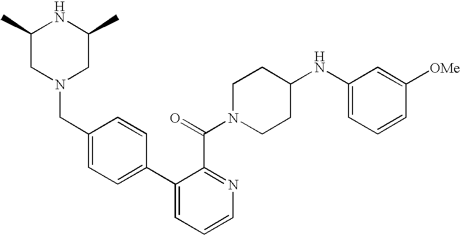 | 1-{[3-(4-{[(3R,5S)-3,5-dimethyl-1-piperazinyl]methyl}phenyl)-2-pyridinyl]carbonyl}-N-[3-(methyloxy)phenyl]-4-piperidinamine | 514 |
| 33 | 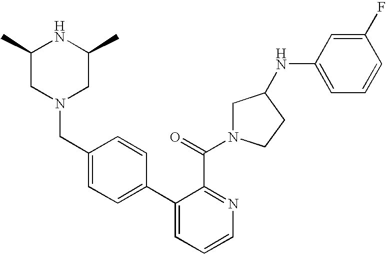 | 1-{[3-(4-{[(3R,5S)-3,5-dimethyl-1-piperazinyl]methyl}phenyl)-2-pyridinyl]carbonyl}-N-(3-fluorophenyl)-3-pyrrolidinamine | 488 |
| 34 | 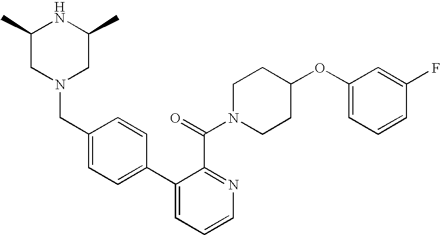 | (3R,5S)-1-({4-[2-({4-[(3-fluorophenyl)oxy]-1-piperidinyl}carbonyl)-3-pyridinyl]phenyl}methyl)-3,5-dimethylpiperazine | 503 |
| 35 | 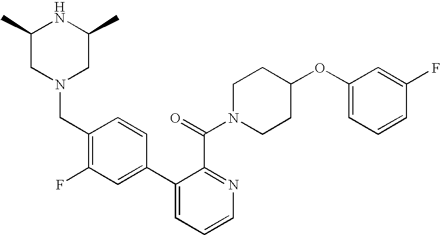 | (3R,5S)-1-({2-fluoro-4-[2-({4-[(3-fluorophenyl)oxy]-1-piperidinyl}carbonyl)-3-pyridinyl]phenyl}methyl)-3,5-dimethylpiperazine | 521 |
| 36 | 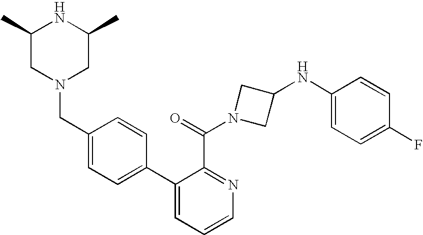 | 1-{[3-(4-{[(3R,5S)-3,5-dimethyl-1-piperazinyl]methyl}phenyl)-2-pyridinyl]carbonyl}-N-(4-fluorophenyl)-3-azetidinamine | 474 |

Compounds of the invention may be tested for in vitro biological activity in accordance with the following FLIPR and GTPγS assays:

### GPR38FLIPR Functional Agonist Assay Protocol

HEK-293 cells stably expressing the GPR38 receptor were seeded (10,000 cells/well) into poly-D-lysine coated 384-well black-wall, clear-bottom microtitre plates (Becton Dickinson) 24 h prior to assay. On day of assay, cells were washed (×2) with 80 ul of assay buffer (Hanks Balanced Salts Solution (HBSS), 10 mM HEPES, 200 μM Ca²⁺, 2.5 mM probenecid) using the EMBLA cell washer. After the final wash, buffer was aspirated to leave a residual volume of 30 ul on the cells. Cells were loaded with 1 μM (final) Fluo-4-AM fluorescent indicator dye (TefLabs) in assay buffer, (20 ul loading solution added to each well using the Multidrop). Plates were incubated for 1 h at 37° C., before being washed (×3) with 80 ul assay buffer using the EMBLA cell washer; 30 ul residual being left after the final wash. Plates were then assayed on a Fluorometric Imaging Plate Reader (FLIPR, Molecular Devices). Test compounds were prepared in assay buffer without probenecid, but containing 0.03% CHAPS. In the FLIPR, 10 ul of test compound was added to the cells and changes in fluorescence measured over a 2 min timeframe. Maximum change in fluorescence over baseline was used to determine agonist response and concentration response curves were constructed, using a 4-parameter logistic equation.

The following alternative procedure may also be used:

HEK-293 cells stably expressing the GPR38 receptor were seeded (30,000 cells/100 ul growth media/well) into poly-D-lysine coated 96-well black-wall, clear-bottom microtitre plates (Corning) 24 hours prior to assay. On the day of assay the cells were loaded with 2 μM (final) Fluo-4-AM fluorescent indicator dye (Molecular Probes) and 1 mM (final) probenicid in assay buffer (145 mM sodium chloride, 2.5 mM potassium chloride, 10 mM Hepes, 10 mM glucose, 1.2 mM magnesium chloride, 1.5 mM calcium chloride and 0.1% BSA) (50 ul loading solution added to each well). Plates were incubated for 1 hour at 25° C., before being washed 4 times with 100 ul assay buffer using the EMBLA cell washer; 150 ul residual being left after the final wash. The cells were then incubated at 25° C. for 20 minutes and the plates were then assayed on a Fluorometric Imaging Plate Reader (FLIPR, Molecular Devices). Test compounds were prepared in assay buffer without probenecid. In the FLIPR, 50 ul of test compound was added to the cells and changes in fluorescence measured over a 2 minute timeframe. Maximum change in fluorescence over baseline was used to determine agonist response and concentration response curves were constructed, using a 4-parameter logistic equation.

Preferred compounds of the invention have a pEC50>5.0 in the FLIPR assay, more preferably >5.5, for example >6.0; The compounds of the examples that have been tested in the FLIPR assay (Examples 1 to 4, 6 to 18, 20, 21, 28, 29, 31 and 33 to 35) have a pEC50>6.0. Examples 1 to 3, 6, 8 to 18, 21, 22, 28, 29 and 33 to 35 had a pEC50>6.5.

### GPR38GTPγS Functional Agonist Assay Protocol

For each compound being assayed, in an Opti clear bottom 96 well plate, is added:—

(a) 20 μl of test compound (or 10 μl of guanosine 5′-triphosphate (GTP) as non-specific binding control) diluted to required concentration in assay buffer (20 mM N-2-Hydroxyethylpiperazine-N′-2-ethanesulfonic acid (HEPES)+100 mM NaCl+10 mM MgCl₂, pH adjusted to 7.4 with NaOH);
(b) 60 μl bead/membrane/GDP mix prepared by suspending wheat germ agglutinin-polyvinyltoluene (WGA-PVT) scintillation proximity assay (SPA) beads at 100 mg/ml in assay buffer followed by mixing with membrane (prepared in accordance with the methodology described above) and diluting in assay buffer to give a final volume of 60 μl which contains 10 μg protein and 0.5 mg bead per well—mixture is pre-mixed at 4° C. for 30 min on a roller and just prior to addition to the plate, 10 μM final concentration of guanosine 5′ diphosphate (GDP—diluted in assay buffer) is added;
(c) 20 μl guanosine 5′ [γ35-S]thiotriphosphate, triethylamine salt (Amersham; radioactivity concentration=37 kBq/μl or 1 mCi/ml; Specific Activity 1160 Ci/mmol) diluted to 1.9 nM in assay buffer to give 0.38 nM final.

The plate is incubated on a shaker at 25° C. for 30 min followed by centrifugation for 5 min at 1500 rpm. The plate is read between 3 and 6 h after completion of centrifuge run in a Wallac Microbeta counter on a 1 min normalised tritium count protocol. Data is analysed using a 4-parameter logistic equation. Basal activity used as minimum.

The following alternative procedure may also be used:

Membranes are derived from bulk cell cultures of HEK293 cell lines transiently transfected with hGPR38R and Go G-protein. P2 membranes fractions are prepared, aliquoted and stored at −80° C.

For each compound being assayed, the following is added into a white Greiner 384 well plate:—

(a) 1 μl of test compound diluted to required concentration in DMSO.
(b) 20 μl bead/membrane/Saponin/GDP mix prepared as follows;—suspension of LEADseeker wheat germ agglutinin (WGA) scintillation proximity assay (SPA) beads at 25 mg/ml in assay buffer (20 mM N-2-Hydroxyethylpiperazine-N′-2-ethanesulfonic acid (HEPES)+100 mM NaCl+10 mM MgCl2, pH adjusted to 7.4 with KOH) containing saponin at 150 ug/ml. Mixing of bead suspension with membranes at 500 ug/ml (prepared in accordance with the methodology described above) and diluting in assay buffer to give a final volume of 20 μl which contains 5 μg protein and 0.25 mg bead per well.

Mixture is pre-mixed for 30 minutes on a roller and just prior to addition to the plate, 3 μM final assay concentration of guanosine 5′ diphosphate (GDP) (diluted in assay buffer) is added.

(c) 25 μl guanosine 5′ [γ35-S]thiotriphosphate, triethylamine salt (Amersham; radioactivity concentration=37 kBq/μl or 1 mCi/ml; Specific Activity 1160 Ci/mmol) diluted to 0.6 nM in assay buffer to give 0.33 nM final assay concentration.

The plate is then spun for 2 minutes at 1500 rpm and then incubated at room temperature for 4 hours. The plate is then read on a Viewlux Plux (Perkin Elmer). Data is analysed using a 4-parameter logistic equation.

Preferred compounds of the invention have a pEC50>5.0 in the GTPγS assay; The compounds of the Examples have been tested in the GTPγS assay and they were found to have a pEC50>5.0. The compounds of Examples 1 to 18, 20 to 25, 27 to 31, 33 and 34 were found to have a pEC50>5.5.

1-27. (canceled)

28. A compound of formula (I) or a pharmaceutically acceptable salt thereof, wherein: 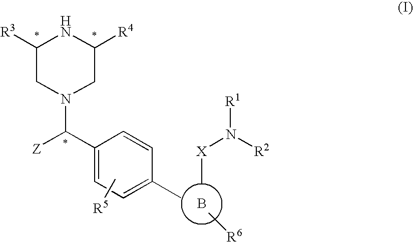
X is CH₂, CO or SO₂;
R³ and R⁴ are independently H or C₍₁₋₄₎alkyl;
R¹ is C₍₁₋₄₎alkyl;
R² is YR⁷;
or R¹ and R² together with the nitrogen to which they are attached form a 4, 5, 6 or 7 membered heterocyclic ring, optionally substituted with one or more substituents independently selected from C₍₁₋₄₎alkyl, hydroxy, ═O or WR⁷;
Y is CO(CH₂)ₙ, SO₂(CH₂)ₙ, (CH₂)ₙ, (CH₂)ₙA, CO(CH₂)ₙA, SO₂(CH₂)ₙA where n is 1, 2, 3 or 4 and A is O, S, CO, SO₂, NH, NHCO, CONH or N—C₍₁₋₄₎alkyl;
W is a bond, CH₂, O, S, CO, SO₂, NH, NHCO, CONH or N—C₍₁₋₄₎alkyl;
R⁷ is optionally substituted phenyl, an optionally substituted 5 or 6 membered heterocyclic ring or an optionally substituted 5 or 6 membered heteroaryl ring;
R⁵ is hydrogen, halogen, or C₍₁₋₄₎alkoxy;
R⁶ is hydrogen, halogen, C₍₁₋₄₎alkyl or C₍₁₋₄₎alkoxy;
Z is H or C₍₁₋₄₎alkyl;
B is a 5 or 6 membered heteroaryl;
and when R⁷ is substituted, it may have 1, 2 or 3 substituents, each independently selected from halogen, C₍₁₋₄₎alkyl, C₍₁₋₄₎alkoxy C₃₋₇cycloalkyl, hydroxy, trifluoromethoxy, trifluoromethyl, nitro, cyano, phenyl, NH₂, NHR⁸NR⁸R⁹, C(O)CF₃, C(O)C₁₋₄alkyl, C(O)C₃₋₇cycloalkyl, CONH₂, CONHR⁸, CONR⁸R⁹, SOR⁹, SO₂R⁹, OSO₂R⁹, OSO₂CF₃, SO₂NH₂, SO₂NHR⁸, SO₂NR⁸R⁹, where R⁸ and R⁹═C₍₁₋₄₎alkyl, phenyl optionally substituted with halogen or 5 or 6 membered heteroaryl optionally substituted with halogen;
but excluding
benzeneacetamide,3,4,5-trimethoxy-N-(2-methylpropyl)-N-[[3-[4 (1 piperazinylmethyl)phenyl]-1,2,4-oxadiazol-5-yl]methyl],
benzeneacetamide,3,4-dichloro-N-(2-methylpropyl)-N-[[3-[4-(1-piperazinylmethyl)phenyl]-1,2,4-oxadiazol-5-yl]methyl],
benzeneacetamide,N-(2-methylpropyl)-α-phenyl-N-[[3-[4-(1-piperazinylmethyl)phenyl]-1,2,4-oxadiazol-5-yl]methyl],
benzenepropanamide,N-(2-methylpropyl)-N-[[3-[4-(1-piperazinylmethyl)phenyl]-1,2,4-oxadiazol-5-yl]methyl],
benzeneacetamide,4-ethoxy-N-(2-methylpropyl)-N-[[3-[4-(1-piperazinylmethyl)phenyl]-1,2,4-oxadiazol-5-yl]methyl],
benzeneacetamide,4-bromo-N-(2-methylpropyl)-N-[[3-[4-(1-piperazinylmethyl)phenyl]-1,2,4-oxadiazol-5-yl]methyl] and
benzeneacetamide,N-(2-methylpropyl)-N-[[3-[4-(1-piperazinylmethyl)phenyl]-1,2,4-oxadiazol-5-yl]methyl]-(trifluoromethyl).

29. A compound according to claim 28 in which, when R⁷ is substituted, has 1, 2 or 3 substituents, each independently selected from halogen, C₍₁₋₄₎alkyl, C₍₁₋₄₎alkoxy, cyano, CONH₂, CONHR⁸, CONR⁸R⁹, SO₂NH₂, SO₂NHR⁸, SO₂NHR⁸R⁹ where R⁸ and R⁹═C₍₁₋₄₎alkyl or optionally substituted phenyl or heteroaryl.

30. A compound according to claim 28 in which X is CH₂ and R² is YR⁷.

31. A compound according to claim 30 in which Y is CO(CH₂)ₙ or CO(CH₂)ₙA and R⁷ is optionally substituted phenyl.

32. A compound according to claim 28 in which R¹ and R² together with the nitrogen to which they are attached form a 5 or 6 membered heterocyclic ring substituted with one or more substituents, one of which is WR⁷.

33. A compound according to claim 28 in which X is CO or SO₂ and R¹ and R² together with the nitrogen to which they are attached form a 4, 5, 6 or 7 membered heterocyclic ring, optionally substituted with one or more substituents, one of which is WR⁷.

34. A compound according to claim 32 in which W is a bond, CH₂, NH, O or CO.

35. A compound according to claim 28 in which R⁷ is optionally substituted phenyl.

36. A compound according to claim 35 in which, when said phenyl is substituted, said substituents are selected from halogen, cyano or CONH₂.

37. A compound according to claim 28 in which NR¹R² is piperidinyl.

38. A compound according to claim 28 in which R¹ is methyl and R² is YR⁷.

39. A compound according to claim 38 in which Y is (CH₂)ₙA or CO(CH₂)ₙA.

40. A compound according to claim 28 in which at least one of R³ and R⁴ does not represent hydrogen.

41. A compound according to claim 28 which is:
(3R,5S)-3,5-dimethyl-1-({4-[2-({4-[(4-fluorophenyl)methyl]piperidin-1-yl}carbonyl)pyridin-3-yl]phenyl}methyl)piperazine;
(3R,5S)-3,5-dimethyl-1-({4-[2-({4-[(4-fluorophenyl)amino]piperidin-1-yl}carbonyl)pyridin-3-yl]phenyl}methyl)piperazine;
N-{[3-(4-{[(3R,5S)-3,5-Dimethyl-1-piperazinyl]methyl}phenyl)-2-pyridinyl]methyl}-3-(4-fluorophenyl)-N-methylpropanamide;
(3R,5S)-3,5-dimethyl-1-({4-[4-({4-[(4-fluorophenyl)methyl]piperidin-1-yl}carbonyl)-2-methyl-1,3-thiazol-5-yl]phenyl}methyl)piperazine;
(3R,5S)-3,5-dimethyl-1-({4-[4-({4-[(4-fluorophenyl)amino]piperidin-1-yl}carbonyl)-2-methyl-1,3-thiazol-5-yl]phenyl}methyl)piperazine;
N-{[5-(4-{[(3R,5S)-3,5-dimethyl-1-piperazinyl]methyl}phenyl)-2-methyl-1,3-thiazol-4-yl]methyl}-3-(4-fluorophenyl)-N-methylpropanamide dihydrochloride;
N-{[5-(4-{[(3R,5S)-3,5-dimethyl-1-piperazinyl]methyl}phenyl)-2-methyl-1,3-thiazol-4-yl]methyl}-2-[(4-fluorophenyl)oxy]-N-methylacetamide;
N-(4-fluorophenyl)-1-{[3-(4-{[(3S)-3-methyl-1-piperazinyl]methyl}phenyl)-2-pyridinyl]carbonyl}-4-piperidinamine;
1-{[3-(4-{[(3R,5S)-3,5-dimethyl-1-piperazinyl]methyl}-3-fluorophenyl)-2-pyridinyl]carbonyl}-N-(4-fluorophenyl)-4-piperidinamine;
1-{[3-(4-{[(3R,5S)-3,5-dimethyl-1-piperazinyl]methyl}phenyl)-2-pyridinyl]carbonyl}-N-(3-fluorophenyl)-4-piperidinamine;
1-{[3-(4-{[(3R,5S)-3,5-dimethyl-1-piperazinyl]methyl}-3-fluorophenyl)-2-pyridinyl]carbonyl}-N-(3-fluorophenyl)-4-piperidinamine;
N-(3,4-difluorophenyl)-1-{[3-(4-{[(3R,5S)-3,5-dimethyl-1-piperazinyl]methyl}-3-fluorophenyl)-2-pyridinyl]carbonyl}-4-piperidinamine;
(3R,5S)-1-({4-[2-({4-[(4-chlorophenyl)thio]-1-piperidinyl}carbonyl)-3-pyridinyl]-2-fluorophenyl}methyl)-3,5-dimethylpiperazine;
N-(3-fluorophenyl)-1-{[3-(4-{[(3S)-3-methyl-1-piperazinyl]methyl}phenyl)-2-pyridinyl]carbonyl}-4-piperidinamine;
N-(3,4-difluorophenyl)-1-{[3-(4-{[(3R,5S)-3,5-dimethyl-1-piperazinyl]methyl}phenyl)-2-pyridinyl]carbonyl}-4-piperidinamine;
1-{[3-(4-{[(3R,5S)-3,5-dimethyl-1-piperazinyl]methyl}phenyl)-2-pyridinyl]carbonyl}-N-(4-fluorophenyl)-3-pyrrolidinamine;
1-({3-[4-{[(3R,5S)-3,5-dimethyl-1-piperazinyl]methyl}-3-(methyloxy)phenyl]-2-pyridinyl}carbonyl)-N-(4-fluorophenyl)-4-piperidinamine;
(3R,5S)-1-({4-[2-({(3S)-3-[(4-fluorophenyl)oxy]-1-pyrrolidinyl}carbonyl)-3-pyridinyl]phenyl}methyl)-3,5-dimethylpiperazine;
(3R,5S)-1-({4-[2-({(3R)-3-[(4-fluorophenyl)oxy]-1-pyrrolidinyl}carbonyl)-3-pyridinyl]phenyl}methyl)-3,5-dimethylpiperazine;
1-{[3-(4-{[(3R,5S)-3,5-dimethyl-1-piperazinyl]methyl}-3-fluorophenyl)-2-pyridinyl]sulfonyl}-N-(2-fluorophenyl)-4-piperidinamine;
1-{[1-(4-{[(3R,5S)-3,5-dimethyl-1-piperazinyl]methyl}phenyl)-1H-imidazol-2-yl]carbonyl}-N-(3-fluorophenyl)-4-piperidinamine;
1-{[1-(4-{[(3R,5S)-3,5-dimethyl-1-piperazinyl]methyl}phenyl)-1H-imidazol-2-yl]carbonyl}-N-(4-fluorophenyl)-4-piperidinamine;
1-{[1-(4-{[(3R,5S)-3,5-dimethyl-1-piperazinyl]methyl}phenyl)-1H-imidazol-2-yl]carbonyl}-N-(2-fluorophenyl)-4-piperidinamine;
1-{[1-(4-{[(3R,5S)-3,5-dimethyl-1-piperazinyl]methyl}-3-fluorophenyl)-1H-imidazol-2-yl]carbonyl}-N-(4-fluorophenyl)-4-piperidinamine;
1-{[1-(4-{[(3R,5S)-3,5-dimethyl-1-piperazinyl]methyl}-3-fluorophenyl)-1H-imidazol-2-yl]carbonyl}-N-(3-fluorophenyl)-4-piperidinamine;
1-{[1-(4-{[(3R,5S)-3,5-dimethyl-1-piperazinyl]methyl}-3-fluorophenyl)-1H-imidazol-2-yl]carbonyl}-N-(2-fluorophenyl)-4-piperidinamine;
N-(3,5-difluorophenyl)-1-{[3-(4-{[(3R,5S)-3,5-dimethyl-1-piperazinyl]methyl}-3-fluorophenyl)-2-pyridinyl]carbonyl}-4-piperidinamine;
1-{[3-(4-{[(3R,5S)-3,5-dimethyl-1-piperazinyl]methyl}-3-fluorophenyl)-2-pyridinyl]carbonyl}-N-[4-fluoro-3-(methyloxy)phenyl]-4-piperidinamine;
3-[(1-{[3-(4-{[(3R,5S)-3,5-dimethyl-1-piperazinyl]methyl}phenyl)-2-pyridinyl]carbonyl}-4-piperidinyl)amino]benzonitrile;
3-[(1-{[3-(4-{[(3R,5S)-3,5-dimethyl-1-piperazinyl]methyl}-3-fluorophenyl)-2-pyridinyl]carbonyl}-4-piperidinyl)amino]benzonitrile;
1-{[3-(4-{[(3R,5S)-3,5-dimethyl-1-piperazinyl]methyl}phenyl)-2-pyridinyl]carbonyl}-N-(2-fluorophenyl)-4-piperidinamine;
1-{[3-(4-{[(3R,5S)-3,5-dimethyl-1-piperazinyl]methyl}phenyl)-2-pyridinyl]carbonyl}-N-[3-(methyloxy)phenyl]-4-piperidinamine;
1-{[3-(4-{[(3R,5S)-3,5-dimethyl-1-piperazinyl]methyl}phenyl)-2-pyridinyl]carbonyl}-N-(3-fluorophenyl)-3-pyrrolidinamine;
(3R,5S)-1-({4-[2-({4-[(3-fluorophenyl)oxy]-1-piperidinyl}carbonyl)-3-pyridinyl]phenyl}methyl)-3,5-dimethylpiperazine;
(3R,5S)-1-({2-fluoro-4-[2-({4-[(3-fluorophenyl)oxy]-1-piperidinyl}carbonyl)-3-pyridinyl]phenyl}methyl)-3,5-dimethylpiperazine; or
1-{[3-(4-{[(3R,5S)-3,5-dimethyl-1-piperazinyl]methyl}phenyl)-2-pyridinyl]carbonyl}-N-(4-fluorophenyl)-3-azetidinamine;
or a pharmaceutically acceptable salt thereof.

42. A pharmaceutical composition comprising a compound according to formula (A) or a pharmaceutically acceptable salt thereof, wherein: 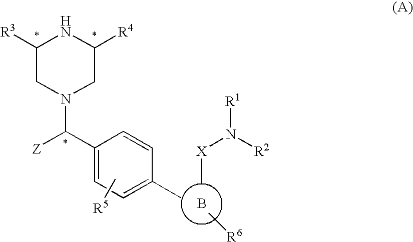
X is CH₂, CO or SO₂;
R³ and R⁴ are independently H or C₍₁₋₄₎alkyl;
R¹ is C₍₁₋₄₎alkyl;
R² is YR⁷;
or R¹ and R² together with the nitrogen to which they are attached form a 4, 5, 6 or 7 membered heterocyclic ring, optionally substituted with one or more substituents independently selected from C₍₁₋₄₎alkyl, hydroxy, ═O or WR⁷;
Y is CO(CH₂)ₙ, SO₂(CH₂)ₙ, (CH₂)ₙ, (CH₂)ₙA, CO(CH₂)ₙA, SO₂(CH₂)ₙA where n is 1, 2, 3 or 4 and A is O, S, CO, SO₂, NH, NHCO, CONH or N—C₍₁₋₄₎alkyl;
W is a bond, CH₂, O, S, CO, SO₂, NH, NHCO, CONH or N—C₍₁₋₄₎alkyl;
R⁷ is optionally substituted phenyl, an optionally substituted 5 or 6 membered heterocyclic ring or an optionally substituted 5 or 6 membered heteroaryl ring;
R⁵ is hydrogen, halogen, or C₍₁₋₄₎alkoxy;
R⁶ is hydrogen, halogen, C₍₁₋₄₎alkyl or C₍₁₋₄₎alkoxy;
Z is H or C₍₁₋₄₎alkyl;
B is a 5 or 6 membered heteroaryl;
and when R⁷ is substituted, it may have 1, 2 or 3 substituents, each independently selected from halogen, C₍₁₋₄₎alkyl, C₍₁₋₄₎alkoxy C₃₋₇cycloalkyl, hydroxy, trifluoromethoxy, trifluoromethyl, nitro, cyano, phenyl, NH₂, NHR⁸, NR⁸R⁹, C(O)CF₃, C(O)C₁₋₄alkyl, C(O)C₃₋₇cycloalkyl, CONH₂, CONHR⁸, CONR⁸R⁹, SOR⁹, SO₂R⁹, OSO₂R⁹, OSO₂CF₃, SO₂NH₂, SO₂NHR⁸, SO₂NR⁸R⁹, where R⁸ and R⁹═C₍₁₋₄₎alkyl, phenyl optionally substituted with halogen or 5 or 6 membered heteroaryl optionally substituted with halogen, and a pharmaceutically acceptable carrier.

43. A process for the preparation of a compound according to formula (I), formula (A) or a pharmaceutically acceptable salt thereof, which comprises:
a) for preparing compounds of formula (I) wherein X═CH₂, a process of reacting a compound of formula (II), 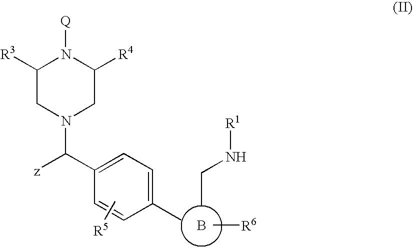
wherein R¹, R³, R⁴, R⁵, R⁶, Z and B are as defined in claim 28, and Q is hydrogen or a nitrogen protecting group,
with a compound of formula (III),
L-Y—R⁷ (III)
wherein Y and R⁷ are as defined in claim 28 and L is a suitable leaving group in the presence of a suitable base, in a suitable solvent;
or
b) for preparing compounds of formula (I) wherein X═CO, a process of reacting a compound of formula (XV), 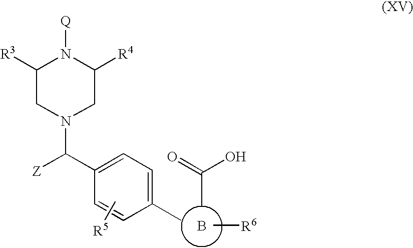
wherein R³, R⁴, R⁵, R⁶, Z and B are as defined in claim 28, and Q is hydrogen or a nitrogen protecting group, with a compound of formula (V),
HNR¹R² (V)
wherein R¹ and R² are as defined in claim 28 with the proviso that when R² is YR⁷ then Y is (CH₂), or (CH₂)ₙA, with a suitable coupling reagent in a suitable solvent;
or
c) for preparing compounds of formula (I) wherein X═SO₂, a process of reacting a compound of formula (XXIV), 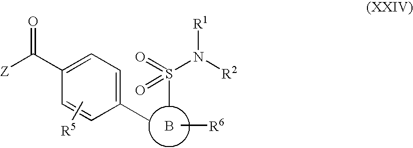
wherein R¹, R², R⁵, R⁶, Z and B are as defined in claim 28 with an appropriately substituted piperazine (X) 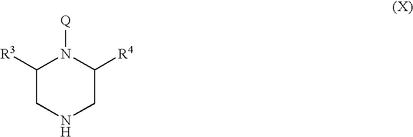
wherein R³ and R⁴ are defined in claim 28 and Q is hydrogen or a nitrogen protecting group, in the presence of a suitable reducing agent, in a suitable solvent;
and in any case (a), (b) or (c) thereafter optionally carrying out one or more of the following reactions:
i) converting one compound of formula (I) into another compound of formula (I);
i) removing any protecting group;
iii) forming a suitable pharmaceutical acceptable salt or solvate of the compound so formed.

44. A process for the preparation of a compound of formula (I) or a pharmaceutically acceptable salt thereof, in which X is CH₂, which comprises reacting a compound of formula (IV): 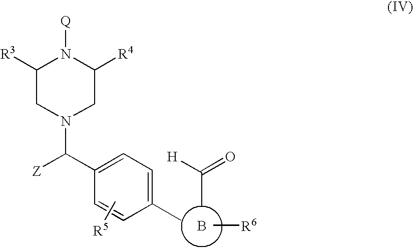
wherein R³, R⁴, R⁵, R⁶, Z and B are as defined in claim 28 and Q is hydrogen or a nitrogen protecting group, with a compound of formula (V),
HNR¹R² (V)
wherein R¹ and R² are as defined in claim 28 with the proviso that when R² is YR⁷, then Y is (CH₂), or (CH₂)ₙA; in the presence of a suitable reducing agent, in a suitable solvent, and thereafter optionally carrying out one or more of the following reactions:
(i) Converting one compound of formula (I) into another compound of formula (I);
(ii) Removing any protecting group;
(iii) Forming a suitable pharmaceutical acceptable salt or solvate of the compound so formed.

45. A process for the preparation of a compound of formula (I) or a pharmaceutically acceptable salt thereof, in which X═CO which process comprises reacting a compound of formula (VIII), 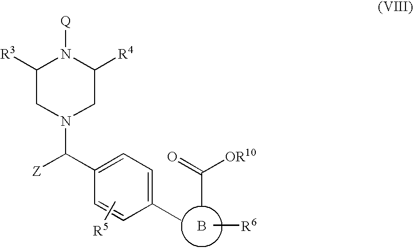
wherein R³, R⁴, R⁵, R⁶, Z and B are as defined in claim 28, R¹⁰ is C₁₋₄alkyl, and Q is hydrogen or a nitrogen protecting group with a compound of formula (V),
HNR¹R² (V)
wherein R¹ and R² are as defined in claim 28 with the proviso that when R² is YR⁷ then Y is (CH₂)ₙ or (CH₂)ₙA, in the presence of trimethylaluminium in a suitable solvent and thereafter optionally carrying out one or more of the following reactions:
(i) Converting one compound of formula (I) into another compound of formula (I);
(ii) Removing any protecting group;
(iii) Forming a suitable pharmaceutical acceptable salt or solvate of the compound so formed.

46. A process for the preparation of a compound of formula (I) or a pharmaceutically acceptable salt thereof, wherein X═CO, which process comprises reacting a compound of formula (XVI), 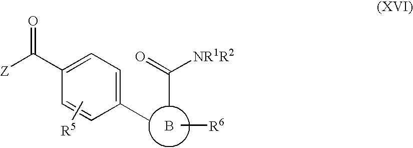
wherein R¹, R², R⁵, R⁶, Z and B are as defined in claim 28, with an appropriately substituted piperazine (X) 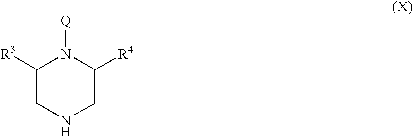
wherein R³ and R⁴ are defined in relation to formula (I) and Q is hydrogen or a nitrogen protecting group, in the presence of a suitable reducing agent, optionally in the presence of a suitable acid catalyst, in a suitable solvent and thereafter optionally carrying out one or more of the following reactions:
(i) Converting one compound of formula (I) into another compound of formula (I);
(ii) Removing any protecting group;
(iii) Forming a suitable pharmaceutical acceptable salt or solvate of the compound so formed.

47. A process for the preparation of a compound of formula (I) or a pharmaceutically acceptable salt thereof, wherein X═SO₂ and R¹, R² and the nitrogen to which they are attached form a piperidine ring substituted by WR⁷, which process comprises reacting a compound of formula (XXVII): 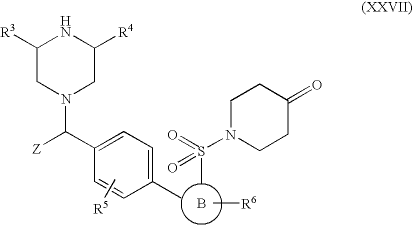
wherein R³, R⁴, R⁵, R⁶, Z and B are as defined in claim 28 with a compound of formula (XXVIII)
HWR⁷ (XXVIII)
wherein W and R⁷ are as defined in claim 28, in the presence of a suitable reducing agent with a suitable acid catalyst in a suitable solvent and thereafter optionally carrying out one or more of the following reactions:
(i) Converting one compound of formula (I) into another compound of formula (I);
(ii) Removing any protecting group;
(iii) Forming a suitable pharmaceutical acceptable salt or solvate of the compound so formed.

48. A compound of formula (II), (XV), (XXIV), (IV), (VIII), (XVI) or (XXVII) in which R¹, R², R³, R⁴, R⁵, R⁶, Z and B are as defined in claim 28, R¹⁰ is C₁₋₄alkyl, and Q is hydrogen or a nitrogen protecting group

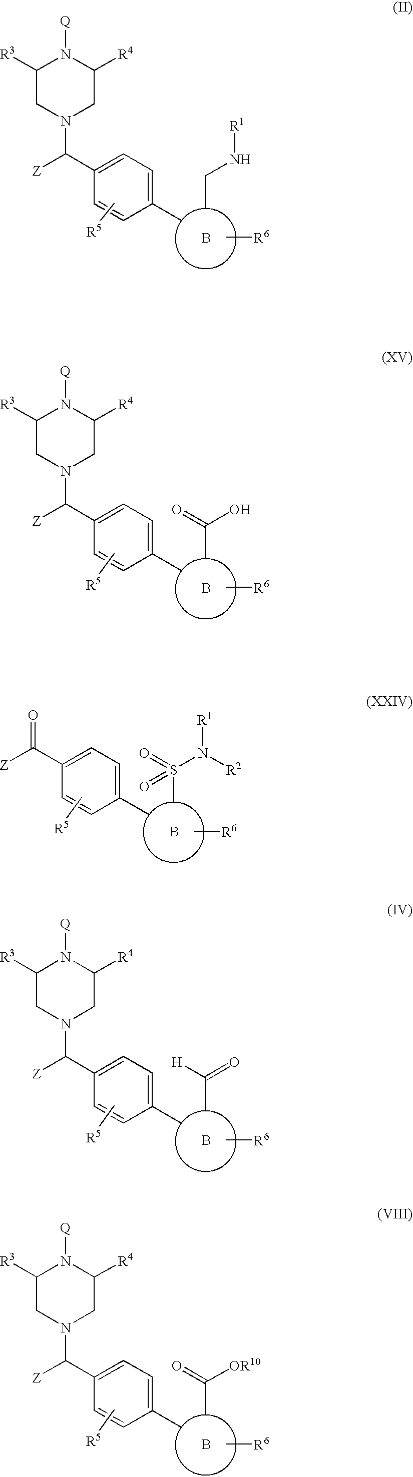

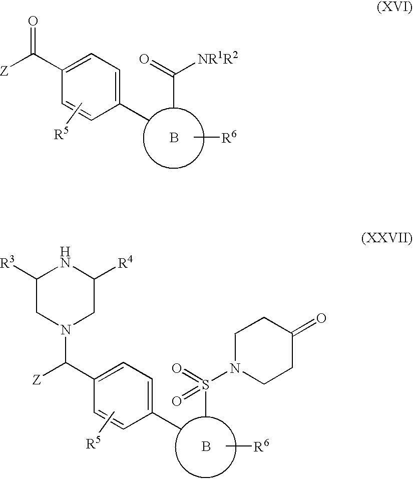

48. A compound of formula (VII), (XXIX) and (XXX) in which R³, R⁴, R⁵, R⁶, Z and B are as defined in claim 28, and Q is hydrogen or a nitrogen protecting group

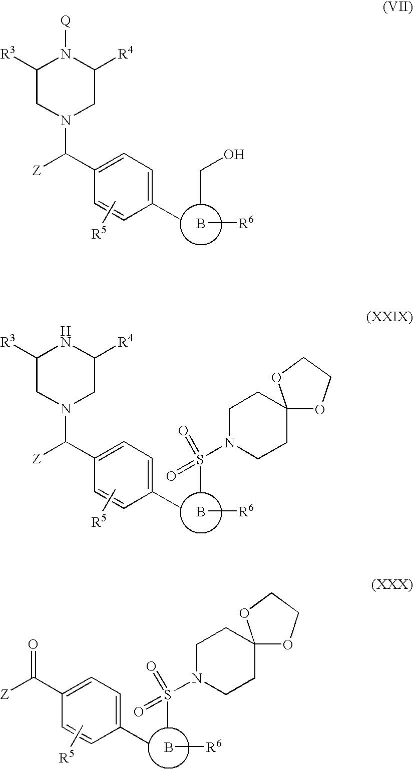

49. A method of treatment of a condition which can be mediated via the GPR³⁸ receptor which comprises administering a compound of formula (I) or formula (A) to a subject in need thereof.

50. The method of claim 49 wherein said condition is a gastrointestinal disorder.

51. The method of claim 49 wherein said condition is selected from gastroesophageal reflux disorders, functional dyspepsia, irritable bowel syndrome, constipation, intestinal pseudo-obstruction, paralytic ileus following surgery or other manipulation, emesis, gastric stasis or hypomotility caused by diabetes and/or by the administration of other drugs, Crohn's disease, colitis, cachexia associated with cancer and/or the treatment thereof, and incontinence.
